(19) United States
(12) Patent Application Publication
(10) Pub. No.: US 2021/0198629 A1
(43) Pub. Date: Jul. 1, 2021

(54) COMPOSITIONS AND METHODS FOR INCREASING BEIGING OF WHITE ADIPOSE TISSUE

(71) Applicant: University of Georgia Research Foundation, Inc., Athens, Georgia 30602-7411, GA (US)

(72) Inventor: Hang Yin, Watkinsville, GA (US)

(21) Appl. No.: 17/269,964

(22) PCT Filed: Aug. 20, 2019
(86) PCT No.: PCT/US2019/047259

§ 371 Date: Feb. 19, 2021

Related U.S. Application Data
(60) Provisional application No. 62/765,312, filed on Aug. 20, 2018.

(51) Int. Cl. C12N 5/077; C07K 14/47; C07K 14/705

(52) U.S. Cl. CPC C12N 5/0653; C07K 14/705; C07K 14/47

(57) ABSTRACT

Compositions and methods for modulating Brd9, Ankib1, Cacng1, and/or Gtl3 (Cfap20) gene or a gene product thereof in a subject in need thereof are disclosed. For example, methods of inducing or increasing white adipose tissue beiging in a subject in need thereof are provided. The methods typically include administering to the subject an effective amount of an inhibitor of Brd9, Ankib1, Cacng1, and/or Gtl3 (Cfap20) gene or a gene product thereof to increase differentiation of white adipose progenitor cells into beige or brown adipose cells. Methods of de-repressing the Pgc1β gene can include administering a subject in need thereof an effective amount of an inhibitor of the Brd9 gene or a gene product thereof to increase expression of the Pgc1β gene in the subject. Treated subjects have or be at risk of developing a metabolic disorder, obesity, reduced endurance or physical activity, muscle loss, or cardiovascular disease.

### CROSS-REFERENCE TO RELATED APPLICATIONS

This application claims the benefit of and priority to U.S. Ser. No. 62/765,312, filed Aug. 20, 2018, which is specifically incorporated by reference herein in its entirety.

### REFERENCE TO THE SEQUENCE LISTING

The Sequence Listing submitted as a text file named “UGA_2018_185_PCT” created on Aug. 12, 2019, and having a size of 11,654 bytes is hereby incorporated by reference pursuant to 37 C.F.R. § 1.52(e)(5).

### FIELD OF THE INVENTION

The invention is generally directed to compositions and methods for modulating pathways important for beiging of white adipose progenitor cells.

### BACKGROUND OF THE INVENTION

Obesity and obesity-associated metabolic syndrome (including high adiposity, hyperglycemia, hypertension, dyslipidemia) are major risk factors for diabetes, cardiovascular diseases, and stroke. A positive energy balance due to overeating and a sedentary lifestyle is a primary cause of current epidemics of obesity and metabolic syndrome. Adipose tissues play crucial roles in modulating whole-body energy homeostasis and glucose metabolism. White adipose tissues (WAT) store caloric energy in the form of triglycerides and release fatty acids in response to fed/fasted states. In contrast, brown adipose tissue (BAT) dissipates caloric energy by characteristic UCP1-dependent uncoupled thermogenesis (non-shivering thermogenesis; NST) under cold exposure. In its activated state, BAT consumes a significant amount of glucose and fatty acids in support of the active heat generation in abundant mitochondria. As such, the thermogenic activity of BAT lowers the food efficiency and hence holds promise to correct the positive energy balance. Indeed, compelling evidence from rodent models indicates that increasing the volume or activity of BAT leads to lean and healthy phenotypes, even with excessive caloric intake (Harms, et al., Nat Med, 19, 1252-1263 (2013)).

BAT is relatively abundant and forms depots in small mammals and human infants (Cannon, et al., Physiol Rev, 84, 277-359 (2004)). However, it was thought that human adults no longer have a physiologically-relevant amount of BAT. Studies using positron emission tomography revealed that BAT activity is prevalent in young and lean human adults (Heaton, J Anat, 112, 35-39 (1972) and Saito, et al., Diabetes, 58, 1526-1531 (2009)). Further characterizations indicate that active human BAT resembles UCP1^{+pos} thermogenic adipocytes (also known as beige or brite adipocytes) that scatter within WATs in rodent models (Wu, et al., Cell, 150, 366-376 (2012) and Sharp, L. Z., et al., PLoS One, 7, e49452 (2012)). In rodents, beige adipocytes emerge in WAT depots upon chronic cold stress, sustained sympathetic stimulation or some disease conditions such as cancer-induced cachexia (Harms, et al., Nat Med, 19, 1252-1263 (2013)). In healthy human adults, the activation of beige adipocytes in WATs by cold exposure or sympathetic stimulants (e.g., adrenergic receptor agonists) increases energy expenditure, glucose tolerance, and insulin sensitivity, which indicates the therapeutic potential of beige adipocytes for obesity and diabetes (Saito, et al., Diabetes, 58, 1526-1531 (2009), 7-9). However, it is also worth noting that BAT mass/activity is low in obese and aged humans (Rogers, N. H., et al., Aging Cell, 11, 1074-1083 (2012) and Berry, D. C., et al., Cell Metab, 25, 166-181 (2017)), who are more likely to be potential patients for BAT-based therapies. Besides, prolonged cold exposure and administration of sympathetic stimulants, though being effective to induce beige adipocyte activation/formation, have risks/side-effects/complications that prevent the therapeutic application of these approaches. Thus, an efficacious and safe means to induce WAT beiging in human adults holds great promise as a treatment for obesity and type 2 diabetes as well as for the prevention of cardiovascular diseases and stroke.

Recent studies in mouse models revealed that beige adipocytes in WATs might develop from two distinct sources. First, prolonged cold exposure induces de novo beige adipogenic differentiation from perivascular white adipose progenitor cells (Berry, D. C., et al., Nat Commun, 7, 10184 (2016); Long, J. Z., et al., Cell Metab, 19, 810-820 (2014); Vishvanath, L., et al., Cell Metab, 23, 350-359 (2016); and Lee, Y. H., et al., Cell Metab, 15, 480-491 (2012)). Without cold stress, these progenitor cells undergo PPARγ-dependent adipogenic differentiation and give rise to white adipocytes during embryonic and postnatal development. After prolonged cold stress, brown/beige-specific lineage determinants (e.g., Ebf2, Prdm16, PGC1α/β transcription factors/cofactors) activate a thermogenic gene program (e.g., UCP1, ElOVL3, DIO2) in some of white adipose progenitor cells, which leads to their differentiation into beige adipocytes (Inagaki, T., et al., Nat Rev Mol Cell Biol, 17, 480-495 (2016)). Intriguingly, human beige adipocytes have been shown to develop from capillary networks within cultured human adipose tissue explants, supporting the above lineage determination model of human beige adipocyte formation (Min, S. Y., et al., Nat Med, 22, 312-318 (2016)). An alternative source of beige adipocytes is terminally differentiated white adipocytes. Recent studies showed that some white adipocytes in mouse models are capable of acquiring the beige adipocyte-specific thermogenesis capacity via a transdifferentiation-like mechanism (white-to-beige adipocyte conversion) upon cold stress or sympathetic simulation (Cinti, S. J Endocrinol Invest, 25, 823-835 (2002); Lee, Y. H., et al., FASEB J, 29, 286-299 (2015); and Rosenwald, M., et al., Nat Cell Biol, 15, 659-667 (2013)). PGC1α/β-dependent mitochondrial biogenesis likely plays an important role in the transformation of mitochondria-less white adipocytes into mitochondria-abundant beige adipocytes (Ikeda, K., et al., Trends Endocrinol Metab, 29, 191-200 (2018)). However, the molecular mechanisms underlying this trans-differentiation type of beige adipocyte formation have not been defined and distinguished with the above-mentioned beige lineage determination mechanisms.

Thus, it is an object of the invention to provide the molecular pathways underlying beige adipocytes formation, and compositions and methods for targeting the pathways.

It is a further object of the invention to provide compositions and methods for increasing beige adipocytes.

### SUMMARY OF THE INVENTION

Compositions and methods for modulating Brd9, Ankib1, Cacng1, and/or Gtl3 (Cfap20) genes and gene products in a subject in need thereof are disclosed. In some embodiments, the Brd9, Ankib1, Cacng1, and/or Gtl3 (Cfap20) gene and/or gene product is a human gene or human gene product. In some embodiments, the subject is human subject.

For example, methods of inducing or increasing white adipose tissue beiging in a subject in need thereof are provided. The methods typically include administering to the subject an effective amount of an inhibitor of a Brd9, Ankib1, Cacng1, and/or Gtl3 (Cfap20) gene or a gene product thereof (e.g., mRNA, protein, etc.) to increase differentiation of white adipose progenitor cells into beige or brown adipose cells.

Methods of de-repressing the Pgc1β gene are also provided. The methods typically include administering a subject in need thereof an effective amount of an inhibitor of, for example, Brd9 gene or a gene product thereof to increase expression of the Pgc1β gene in the subject.

Methods of increasing mitochondrial biogenesis are provided. The methods typically includes administering a subject in need thereof an effective amount of an inhibitor of a Brd9, Ankib1, Cacng1, and/or Gtl3 (Cfap20) gene or a gene product thereof to increase mitochondrial biogenesis in the subject. In some embodiments, expression of the mitochondrial marker Cox8b is increased.

In some embodiments of the disclosed methods, the subject has or is at risk of developing a metabolic disorder, obesity, reduced endurance or physical activity, muscle loss, or cardiovascular disease. Thus, also disclosed are methods of treating a condition, disorder, or disease in a subject. The methods typically include administering a subject in need thereof an effective amount of an inhibitor of Brd9, Ankib1, Cacng1, and/or Gtl3 (Cfap20) gene or a gene product thereof to treat one or more symptoms of the condition, disorder, or disease, particularly those in which the condition, disorder, or disease is a metabolic disorder, obesity, reduced endurance or physical activity, muscle loss, or cardiovascular disease. The metabolic disorder can be, for example, insulin resistance, Type 1 or 2 diabetes mellitus, insulin insensitivity, impaired fasting glycaemia, impaired glucose tolerance (IGT), dysglycemia, dyslipidemia or metabolic syndrome.

In some embodiments, the inhibitor is administered in an effective amount to increase expression of one or more beige lineage markers. Beige lineage markers include, for example, Pgc1α, Pgc1β, Ucp1, Cedia, Dio2, Elovl3, Cox8b, and combinations thereof. In some embodiments, the inhibitor is administered in an effective amount to increase one or more markers of mitochondrial biogenesis. Markers of mitochondrial biogenesis include, for example, Pgc1β, Ndufb2, Sdha, Uqcrc2, Cox8b, Atp5a1, copies of mitochondrial genomic DNA, and combinations thereof. In some embodiments, the inhibitor induces or increases weight loss, prevents weight gain, reduces fat mass, increases lean mass, increases energy expenditure, increases time to exhaustion, increases oxygen consumption, improves β-cell function, improves insulin resistance, improves glucose tolerance, improves insulin sensitivity, or a combination thereof in the subject.

Exemplary inhibitors are also provided. For example, in some embodiments, the inhibitor is antisense, siRNA, miRNA, aptamers, ribozymes, triplex forming molecules, RNAi, external guide sequences, or a gene editing composition that targets a Brd9, Ankib1, Cacng1, and/or Gtl3 (Cfap20) gene or a gene product thereof. The gene editing composition can be one that induces a single or double strand break at a Brd9, Ankib1, Cacng1, and/or Gtl3 (Cfap20) genetic locus in the subject and reduces expression thereof. An exemplary gene editing composition is a CRISPR/Cas system. The CRISPR/Cas system can include, for example, a single-guide RNA (sgRNA) that targets a Brd9, Ankib1, Cacng1, and/or Gtl3 (Cfap20) and Cas nuclease or nickase.

In some embodiments, the inhibitor is a pharmacological inhibitor such as a small molecule inhibitor.

In particularly preferred embodiments, the inhibitor is an inhibitor of a Brd9 gene or gene product thereof. Exemplary pharmacological inhibitors of Brd9 genes or gene products are also provided. Such inhibitors include, for example, LP99, I-BRD9, BI-7273, BI-9564, GNE-375, and combinations thereof. An exemplary combination is BI-7273 or BI-9564 in combination with I-BRD9.

In some preferred embodiments, the inhibitor is an inhibitor of the Cacng1 gene or gene product thereof. Pharmacological inhibitors of Cacng1 genes or gene products include, but are not limited to, dihydropyridine (DHP) calcium channel blocker such as amlodipine, aranidipine), azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, or pranidipine.

Pharmaceutical compositions including an effective amount of one or more Brd9, Ankib1, Cacng1, and/or Gtl3 (Cfap20) inhibitors to treat a subject in need thereof are also provided. In some embodiments the inhibitor is effective to induce or increase weight loss, prevent weight gain, reduce fat mass, increases lean mass, increase energy expenditure, increase time to exhaustion, increase oxygen consumption, improve β-cell function, improve insulin resistance, improve glucose tolerance, improve insulin sensitivity, or a combination thereof in a subject.

### BRIEF DESCRIPTION OF THE DRAWINGS

FIG. 1A is a diagram showing the steps in a screen of beige adipocyte lineage repressors using white adipose progenitor cells and a fluorescent reporter (Ucp1-Cre;LSL-nmGFP). FIG. 1B is representative differential interference contrast (DIC) and fluorescence images of nmGFP^{+pos} beige adipocytes in a culture of iWAT progenitor cells differentiated under a pro-beiging adipogenic differentiation condition with rosiglitazone (Rosi.) and T3 thyroid hormone (T3). Notably, beige adipocytes have GFP fluorescence on the nuclear membrane. FIG. 1C is representative FACS profiles and gates used to identify nmGFP^{+pos} beige adipocytes from primary adipocyte cultures. Primary adipocytes were derived from progenitor cells that were isolated from iBAT, iWAT and eWAT depots of Ucp1-Cre;ROSA-LSL-nmGFP mice and differentiated under a pro-beiging adipogenic differentiation condition with Rosi. and T3. Notably, GFP vs. PE signals was plotted to distinguish nmGFP^{+pos} beige adipocytes (gated) vs. nmGFP^{-neg} cells (on the diagonal line) that may have strong autofluorescence. FIG. 1D is a bar graph showing RT-qPCR of Ucp1 mRNA levels in the FACS-sorted nmGFP^{+pos} beige adipocytes and GFP^{-neg} fractions. FIG. 1E is the actual FACS profiles (and gates) of primary adipocytes (derived from progenitor cells from iWAT or eWAT depots) in the primary screening. Statistical significance was determined by Prism GraphPad 6 using unpaired two-tailed Student's t-test. *: p<0.05, **: p<0.01, n.s.: not significant.

FIG. 2A is a series of representative DIC and immunofluorescence images of primary adipocyte cultures in the secondary screening. Perilipin is a marker for oil droplets in differentiated adipocytes. Notably, primary adipocytes with Brd9, Ankib1, Cacng1 and Gtl3 knockout had comparable numbers of Perilipin^{+pos} oil droplets compared to the control cells. FIGS. 2B-2E are bar graphs showing the results of RT-qPCR of mRNA levels of beige adipocyte markers (Pgc1α, Ucp1, Cedia, Dio2, Elovl3, Cox8b) in primary adipocytes with individual Brd9, Ankib1, Cacng1 and Gtl3 knockout. FIG. 2F is a bar graph showing the results of RT-qPCR of the beige adipocyte markers in primary adipocytes with the knockout of a non-beige lineage determinant FIGS. 2G-2J are bar graphs showing the results of RT-qPCR of mRNA levels of beige adipocyte markers (Pgc1α, Ucp1, Cedia, Dio2, Elovl3, Cox8b) in iWAT and eWAT with individual Brd9, Ankib1, Cacng1 and Gtl3 knockout. Statistical significance was determined by Prism GraphPad 6 using unpaired two-tailed Student's t-test. *: p<0.05, **: p<0.01, n.s.: not significant.

FIGS. 3A-3C are bar graphs showing the results of RT-qPCR of beige lineage markers, adipocyte markers and mitochondrial markers in adipocyte cultures that were treated with BI-7273 or vehicle (DMSO) in the progenitor stage for 48 hrs and later differentiated under the basal differentiation condition. FIGS. 3D-3F are bar graphs showing the results of RT-qPCR of beige lineage markers, adipocyte markers and mitochondrial markers in adipocyte cultures that were first differentiated under the basal differentiation condition and then treated with BI-7273 or vehicle (DMSO) for 48 hrs. FIGS. 3G-3I are bar graphs showing the results of RT-qPCR of beige lineage markers, adipocyte markers and mitochondrial markers in adipocyte cultures that were treated with BI-9564 or control chemical (BI-6354) in the progenitor stage for 48 hrs and later differentiated under the basal differentiation condition. FIG. 3J is a bar graph showing the result of ChIP-qPCR of BRD9. FIG. 3K is a bar graph showing ChIP-qPCR of BI-7273 treated white adipose progenitor cells. FIG. 3L is a bar graph showing the results of RT-qPCR of Pgc1β in BI-7273 treated and vehicle treated white adipose progenitor cells. Statistical significance was determined by Prism GraphPad 6 using unpaired two-tailed Student's t-test. *: p<0.05, **: p<0.01, n.s.: not significant.

FIGS. 4A-4B are image of H/E staining and UCP1 IHC of iWAT (4A) or eWAT (4B) from high-fat diet fed C57BL/6J mice (DIO mice) orally administered with BI-7273 or the vehicle (0.5% Natrosol) for 14 days. FIGS. 4C-4D are bar graphs showing the results of RT-qPCR of beige lineage markers in iWAT (4C) or eWAT (4D) from DIO mice administered with BI-7273 or the vehicle (0.5% Natrosol) for 14 days. FIG. 4E is a bar graph showing the results of RT-qPCR of Pgc1β in multiple tissues from DIO mice administered with BI-7273 or the vehicle (0.5% Natrosol) for 14 days. FIG. 4F is a bar graph showing the result of qPCR of mitochondrial genome copy numbers in multiple tissues from DIO mice administered with BI-7273 or the vehicle (0.5% Natrosol) for 14 days. Statistical significance was determined by Prism GraphPad 6 using unpaired two-tailed Student's t-test. *: p<0.05, **: p<0.01, n.s.: not significant.

FIGS. 5A-5C are bar graphs showing Energy expenditure (EE) (5A), VO₂ (5B), RER (5C). FIG. 5D is a dot plot showing physical activity (5D) of DIO mice administered with BI-7273 or the vehicle (0.5% Natrosol) for 14 days. Data of light and dark cycles during the 7-day indirect calorimetry measurement were separately presented. FIG. 5E is a representative image of direct comparison of DIO mice administered with BI-7273 or the vehicle (0.5% Natrosol) for 14 days. FIG. 5F is a line graph showing the averaged body weight of DIO mice after 2 months of HFD feeding (0 weeks), during BI-7273/0.5% Natrosol gavaging (0-2 weeks, on normal chow), and during 1 month of HFD refeeding (2-6 weeks). FIG. 5G-5I are bar graphs showing the Fat mass percentages (5G), food intake (5H), and treadmill performance (5I) of DIO mice before (as 0 weeks in panel 5F) and after (as 6 week in panel 5F) BI-7273 treatment. Statistical significance was determined by Prism GraphPad 6 using unpaired two-tailed Student's t-test. *: p<0.05, **: p<0.01, n.s.: not significant.

FIG. 6A-6C are bar graphs showing blood glucose (6A), HbA1c (6B), and HOMA-IR index (6C) of DIO mice administered with BI-7273 or the vehicle (0.5% Natrosol) for 14 days. Before treatment: after 2-month HFD feeding and before gavage. Post-treatment: after 2-week gavage and additional 1-month HFD refeeding. FIGS. 6D-6E are line graphs showing IPGTT (6D) and IPITT (6E) of DIO mice that have been orally administered with BI-7273 or the vehicle (0.5% Natrosol) for 14 days and refed with HFD for 1 month. Statistical significance was determined by Prism GraphPad 6 using unpaired two-tailed Student's t-test. *: p<0.05, **: p<0.01, n.s.: not significant.

### DETAILED DESCRIPTION OF THE INVENTION

### I. Definitions

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “inhibit” or other forms of the word such as “inhibiting” or “inhibition” means to hinder or restrain a particular characteristic. It is understood that this is typically in relation to some standard or expected value, in other words it is relative, but that it is not always necessary for the standard or relative value to be referred to. For example, “inhibits BRD9” means hindering or restraining the activity of the protein relative to a standard or a control. “Inhibits BRD9” can also mean to hinder or restrain the synthesis or expression of the protein, or mRNA encoding the protein, relative to a standard or control.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, the term “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “localization signal or sequence or domain or ligand” or “targeting signal or sequence or domain or ligand” are used interchangeably and refer to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location.

As used herein, the term “microparticles” refers to particles having a diameter between one micron and 1000 microns, typically less than 400 microns, more typically less than 100 microns, most preferably for the uses described herein in the range of less than 10 microns in diameter. Microparticles include microcapsules and microspheres unless otherwise specified.

As used herein, the term “nanoparticles” refers to particles having a diameter of less than one micron, more typically between 50 and 1000 nanometers, preferably in the range of 100 to 300 nanometers.

As used herein, “age-related disorder” includes any disease, disorder, or condition associated with aging or increased age, and includes, but is not limited to, all of the diseases, disorders and conditions described herein. A subject of any age can suffer from, or be diagnosed with an age-related disorder.

As used herein a human “newborn” is less than 1 month old, an “infant” is about 1 month to about 12 months old; a “child” is about 1 year to about 12 years old; an “adolescent” is about 13 years to about 17 years old; an “adult” is about 18 years to about 64 years old; and an “elder” or “elderly person” (also referred to collectively as “the elderly”) is greater than about 64 years old. As used herein, “treat” means to prevent, reduce, decrease, or ameliorate one or more symptoms, characteristics or comorbidities of an age-related disease, disorder or condition; to reverse the progression of one or more symptoms, characteristics or comorbidities of an age related disorder; to halt the progression of one or more symptoms, characteristics or comorbidities of an age-related disorder; to prevent the occurrence of one or more symptoms, characteristics or comorbidities of an age-related disorder; to inhibit the rate of development of one or more symptoms, characteristics or comorbidities or combinations thereof.

As used herein “comorbidity” means one or more disorders or diseases in addition to the age-related disease or disorder of interest, or an effect of such additional disorders or diseases.

As used herein, the term “eukaryote” or “eukaryotic” refers to organisms or cells or tissues derived therefrom belonging to the phylogenetic domain Eukarya such as animals (e.g., mammals, insects, reptiles, and birds), ciliates, plants (e.g., monocots, dicots, and algae), fungi, yeasts, flagellates, microsporidia, and protists.

As used herein, the term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, the term “vector” refers to a replicon, such as a plasmid, phage, or cosmid, into which another DNA segment may be inserted so as to bring about the replication of the inserted segment. The vectors can be expression vectors.

As used herein, the term “expression vector” refers to a vector that includes one or more expression control sequences.

As used herein, the term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, the terms “transformed,” “transgenic,” “transfected” and “recombinant” refer to a host organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

As used herein, the term “endogenous” with regard to a nucleic acid refers to nucleic acids normally present in the host.

As used here, the term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

As used herein, “substantially changed” means a change of at least e.g. 5%, 10%, 15%, 20%, 25%, 30%, 35%, 40%, 45%, 50%, 75%, 100%, or more relative to a control.

### II. Method of Use

White adipose tissues (WAT) store caloric energy in the form of triglycerides and release fatty acids in response to fed/fasted states, while activated brown adipose tissue (BAT) can consume glucose and fatty acids in support of the active heat generation and thus dissipate caloric energy. Thus, decreasing WAT and/or increasing BAT cells has implications in a host of diseases and disorders ranging from obesity and type 2 diabetes to cardiovascular diseases and stroke. The studies described in the Examples below identify Brd9, Ankib1, Cacng1, and Gtl3 (Cfap20) beige adipocyte lineage repressors in white adipose progenitor cells induced beige adipocyte differentiation in vitro and in vivo. Thus, modulation of Brd9, Ankib1, Cacng1, and Gtl3 (Cfap20) genes and gene products thereof can be used to modulate the WAT and BAT content of adipose tissues.

Compositions and methods for inducing or increasing WAT beiging in a subject in need thereof are provided. Typically, the compositions and methods reduce or remove the repressive effects of a gene product of Brd9, Ankib1, Cacng1, and/or Gtl3 (Cfap20). For example, the composition can reduce or prevent gene expression of Brd9, Ankib1, Cacng1, and/or Gtl3 (Cfap20), or reduce or otherwise inhibit an activity a gene product thereof. In some embodiments, the subject has is at risk of developing diabetes, insulin resistance, muscle atrophy, cardiovascular disease or a combination thereof. In some embodiments, the composition can induce or increase weight loss, or delay or decrease weight gain. Thus, in some embodiments, the subject is overweight or obese. In some embodiments, the composition is administered to reduce or prevent one or more effects of aging. As discussed in more detail below, the compositions can be administered to a subject in need thereof in an effective amount to reduce one or more symptoms of the disease or disorder or condition to be treated, to reduce one or more biochemical or cellular markers associated with WAT, increase one or more biochemical or cellular marker associated with BAT, or a combination thereof.

It will be appreciated that active human BAT resembles UCP1^{+pos} thermogenic adipocytes (also known as beige or brite adipocytes) that scatter within WATs in rodent models. Thus, the terms BAT, beiging or beige WAT, and thermnogenic adipocytes can be used interchangeable with respect the compositions and methods of use disclosed herein.

A. Methods of Treatment

The disclosed methods typically includes administering a subject in need thereof an effective amount of a composition that increases BAT in a subject in need thereof. In some embodiments, the composition increases BAT by inducing or increasing beiging WAT progenitor cells. Thus, in some embodiments, differentiated BAT is increased and differentiated WAT is decreased. Most typically, the compositions and methods directly or indirectly reduce or otherwise inhibit the level of gene or mRNA expression of Brd9, Ankib1, Cacng1, and/or Gtl3 (Cfap20), or the expression, localization, or activity of a protein encoded by Brd9, Ankib1, Cacng1, and/or Gtl3 (Cfap20).

In some embodiments, the effect of the disclosed compositions and methods on a subject is compared to a control. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art, such as one of those discussed herein.

In some embodiments, the composition reduces or removes the repressive effects of a gene product (e.g., mRNA and/or protein) of Brd9, Ankib1, Cacng1, and/or Gtl3 (Cfap20) gene locus also referred to herein as “a beige lineage repressors” in some embodiments, Brd9, Ankib1, Cacng1, and/or Gtl3 (Cfap20) gene products can repress beige adipocyte lineage differentiation, repress the white-to-beige adipocyte conversion, or a combination thereof. Limiting or preventing repression caused by the gene products encoded by Brd9, Ankib1, Cacng1, and/or Gtl3 (Cfap20) thus increases, enhances, or induces beige adipocyte lineage differentiation, white-to-beige adipocyte conversion, or a combination thereof. In some embodiments, reducing or inhibiting Brd9, Ankib1, Cacng1, and/or Gtl3 (Cfap20) or a gene product thereof in white adipose progenitor cells increases beige adipogenic differentiation. Thus in some embodiments, beiging of WAT progenitor cells is increased. In some embodiments, the composition additionally or alternatively enhances, increases, or improves differentiation of BAT progenitor cells into differentiated BAT, or maintains differentiated BAT in a BAT-like state.

In some embodiments the composition induces the beige lineage determination and differentiation of white adipose progenitor cells, without substantially increasing white-to-beige adipocyte conversion, and/or without repressing the overall differentiation of white beige adipocytes per se. Thus, in some embodiments, the compositions drive WAT progenitor cells toward a beige, thermogenic phenotype at the expense of a white phenotype. Thus, in some embodiments, notwithstanding a change of differentiation in favor of beiging, the number of Perilipin^{+pos} multiocular adipocytes is not substantially increased.

The WAT progenitor cells can be in subcutaneous WAT [such as inguinal WAT depots (iWAT)], intra-abdominal WAT [such as epidydimal WAT depots (eWAT)], or a combination thereof. The depots can be anywhere in the subject. For example, humans have three main regional anatomical fat depots: intra-abdominal, upper-body/abdominal subcutaneous, and lower body subcutaneous. Subcutaneous and intra-abdominal can include all major fat depots. White adipocytes can also be found in perivescular regions, epicardial regions, skeletal muscle, bone marrow, and breast. In specific embodiments, the depots are in one or more limb muscles and/or the liver. In some embodiments the BAT is from interscapular, supraclavicular, and paraspinal BAT depots (iBAT).

The induced or increased beiging WAT can express one or more beige lineage markers, or otherwise indicate the emergence of a phenotype characteristic of thermogenic beige adipocytes. Thus, in some embodiments, the compositions are effective to increase mRNA and/or protein levels of beige lineage markers including, but not limited to, Pgc1α, Ucp1, Cedia, Dio2, Elovl3, Cox8b, or a combination thereof.

In some embodiments, the composition does not substantial change the expression of one or more adipocyte markers, for example, Pparg2, Leptin, Adiponectin, and Fabp4. In some embodiments, the composition induces or increases beige adipocyte formation from white adipose progenitor cells without the aid of other pro-beiging compounds. Other brown/beige-specific lineage determinants include, for example, Ebf2, Prdm16, PGC1α/β transcription factors/cofactors.

In some embodiments, the composition does increases the expression of one or more adipocyte markers, for example, Pparg2, Leptin, Adiponectin, and Fabp4.

Additionally or alternatively the compositions can increase mitochondrial biogenesis or a phenotype characteristic thereof. Thus, in some embodiments, the compositions are effective to increase mRNA and/or protein levels of mitochondrial markers, or markers of biogenesis thereof, including, but not limited to, Pgc1β, Ndufb2, Sdha, Uqcrc2, Cox8b, Atp5a1, or a combination thereof. In some embodiments, the number of mitochondria and/or number of copies of mitochondrial DNA is increased.

In particular embodiments, the composition is effective to reduce the overall size of adipocytes, induce the formation of multiocular UCP1-expressing beige adipocytes, or a combination thereof.

The Examples below also illustrated that at least some of the repressors of beige adipocyte lineage differentiation repress expression of Pgc1β. For example, the experiments below illustrate that BRD9 directly associates with the proximal promoter of Pgc1β gene, and sgRNA-mediated Brd9 genetic ablation increases the Pgc1β mRNA levels in multiple tissues (adipose tissues, skeletal muscle, heart, and liver). These data indicate that BRD9 represses the beige adipocyte lineage determination by repressing PGC1β expression. However, PGC1β has also been shown to induce mitochondrial biogenesis in skeletal muscle and heart; conversely, a repressed PGC1β expression is a fundamental cause of pressure overload-induced heart hypertrophy and heart failure. Thus, de-repression of PGC1β expression, by, for example, inhibiting BRD9 expression or activity can be effective for treating a variety of conditions including, but not limited to, aging, muscle atrophy, and heart failure. Such treatment may be dependent on or independent of effects of the composition on adipocyte lineage determination.

Thus, in some embodiments, the compositions and methods are effective to reduce one or more symptoms of a disease, disorder, and condition to be treated. Such diseases, disorders, and conditions and more specific symptoms thereof are discussed in more detail below.

More generally, in some embodiments, the compositions can induce or increase weight loss, prevent weight gain, reduce fat mass, increase lean mass, increase energy expenditure (e.g., total energy expenditure (E.E.)), increase time to exhaustion (e.g., during physical activity including, but not limited to, exercise), increase oxygen consumption (e.g., (V02)), improve (3-cell function, improve insulin resistance (e.g., reduce insulin resistance), improve glucose tolerance, improve insulin sensitivity, or a combination thereof in subject.

The route of administration can be oral, parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In certain embodiments, the compositions are administered locally, for example by injection or other application directly into or onto a site to be treated. In some embodiments, the compositions are injected, topically applied, or otherwise administered directly into adipose tissue. Typically, local administration causes an increased localized concentration of the compositions which is greater than that which can be achieved by systemic administration.

In some embodiments the route of administration is transdermal, for example, a transdermal patch or gel that is contacted with the skin of the subject. The composition can be administered directly or indirectly to adipose tissue and/or the target cells (e.g., WAT precursor cells).

The precise dosage will vary according to a variety of factors including but not limited to the inhibitor that is selected and subject-dependent variables (e.g., age, immune system health, clinical symptoms etc.).

In cases of a solid dosage form, examples of daily dosages of the compounds described herein which can be used are an effective amount within the dosage range of about 0.001 mg to about 2 mg per kilogram of body weight, about 0.001 mg to about 5 mg per kilogram of body weight, about 0.001 mg to about 10 mg per kilogram of body weight, about 0.001 mg to about 20 mg per kilogram of body weight, about 0.001 mg to about 50 mg per kilogram of body weight, about 0.001 mg to about 100 mg per kilogram of body weight, about 0.001 mg to about 200 mg per kilogram of body weight, or about 0.001 mg to about 300 mg per kilogram of body weight.

When administered orally or by inhalation, examples of daily dosages are an effective amount within the dosage range of about 0.1 mg to about 10 mg, or about 0.1 mg to about 20 mg, or about 0.1 mg to about 30 mg, or about 0.1 mg to about 40 mg, or about 0.1 mg to about 50 mg, or about 0.1 mg to about 60 mg, or about 0.1 mg to about 70 mg, or about 0.1 mg to about 80 mg, or about 0.1 mg to about 90 mg, or about 0.1 mg to about 100 mg, or about 0.1 mg to about 200 mg, or about 0.1 mg to about 300 mg, or about 0.1 mg to about 400 mg, or about 0.1 mg to about 500 mg, or about 0.1 mg to about 600 mg, or about 0.1 mg to about 700 mg, or about 0.1 mg to about 800 mg, or about 0.1 mg to about 900 mg, or about 0.1 mg to about 1 g, or about 20 mg to 300 mg, or about 20 mg to 500 mg, or about 20 mg to 700 mg, or about 20 mg to 1000 mg, or about 50 mg to 1500 mg, or about 50 mg to 2000 mg.

Exemplary fixed daily doses include about 1 mg, about 2 mg, about 3 mg, about 4 mg, about 5 mg, about 6 mg, about 7 mg, about 8 mg, about 9 mg, about 10 mg, about 12 mg, about 15 mg, about 18 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1200 mg, about 1500 mg, or about 2000 mg, independently of body weight. However, it is understood that pediatric patients may require smaller dosages, and depending on the severity of the disease and condition of the patient, dosages may vary.

When formulated as a liquid, the concentration of the compounds described herein may be about 0.01 mg/ml to about 0.1 mg/ml or about 0.1 mg/ml to about 1 mg/ml, but can also be about 1 mg/ml to about 10 mg/ml or about 10 mg/ml to about 100 mg/ml. The liquid formulation could be a solution or a suspension. When formulated as a solid, for example as a tablet or as a powder for inhalation, the concentration, expressed as the weight of a compound divided by total weight, will typically be about 0.01% to about 0.1%, about 0.1% to about 1%, about 1% to about 10%, about 10% to about 20%, about 20% to about 40%, about 40% to about 60%, about 60% to about 80%, or about 80% to about 100%.

The timing of the administration of the composition will also depend on the formulation and/or route of administration used. The compound may be administered once daily, but may also be administered two, three or four times daily, or every other day, or once or twice per week. For example, the subject can be administered one or more treatments 1, 2, 3, 4, 5, 6, 7, 8, 9, 10, 11, 12, 13, 14, 15, 16, 17, 18, 19, 20, 21, 22, 23, or 24 hours, days, weeks, or months apart.

In some embodiments, the compositions are formulated for extended release. For example, the formulation can be suitable for administration once daily or less. In some embodiments, the composition is only administered to the subject once every 24-48 hours.

In some embodiments, administration of the composition will be given as a long-term treatment regimen whereby pharmacokinetic steady state conditions will be reached.

B. Conditions to be Treated

The disclosed compositions and methods are particularly useful for treating a subject having a disease, disorder, condition or symptom or comorbidity thereof associated with aging or increased age, metabolic disorders such as insulin resistance or diabetes, vascular disease, heart disease, atherosclerosis, dyslipidemia, liver steatosis, obesity or excessive weight gain, loss of physical activity, loss of endurance, loss of skeletal muscle strength, and loss of skeletal muscle mass. In some embodiments, the compositions and methods can reduce aging or pre-mature aging, increase longevity, increase lifespan, or combination thereof in a subject. The compositions can be used to increase mitochondrial biogenesis and oxidative metabolism.

1. Metabolic Disorders

The disclosed compositions and methods are useful for treating one or more symptoms or comorbidities of a metabolic disorder, including, but not limited to, insulin resistance, Type 1 or 2 diabetes mellitus, insulin insensitivity, impaired fasting glycaemia, impaired glucose tolerance (IGT), dysglycemia, dyslipidemia, hypertriglyceridemia, hyperglyceridemia, dyslipoproteinemia, hyperlipidemia, hypercholesterolemia, hypolipoproteinemia, and metabolic syndrome.

a. Insulin Resistance and Diabetes

In some embodiments the disclosed compositions are used to treat or prevent insulin resistance or diabetes. Insulin resistance and diabetes can be diagnosed using an oral glucose tolerance test (OGTT). Typically, a fasting patient takes a 75 gram oral dose of glucose. Blood glucose levels are then measured over the following 2 hours. After 2 hours a glycemia less than 7.8 mmol/L (140 mg/dl) is considered normal, a glycemia of between 7.8 to 11.0 mmol/dl (140 to 197 mg/dl) is considered as Impaired Glucose Tolerance (IGT) and a glycemia of greater than or equal to 11.1 mmol/dl (200 mg/dl) is considered diabetes mellitus. An OGTT can be normal or mildly abnormal in simple insulin resistance. A fasting serum insulin level of greater than approximately 60 pmol/L is also considered evidence of insulin resistance.

In some embodiments, the disclosed compositions reduce or decrease fasting blood glucose level, insulin level, or combinations thereof, or to reduce, decrease, or delay a rise in fasting blood glucose level, insulin level, or combinations thereof over time. In some embodiments, the compositions disclosed herein delay a rise in fasting blood glucose level, insulin level, or combinations thereof that can occur over time in subjects with high fat diets, little or no exercise, hereditary mutations, hormone changes, advanced age (i.e., becoming elderly), increasing weight or other factors that put them at risk for insulin resistance or diabetes.

b. Metabolic Syndrome

In some embodiments the metabolic disorder is metabolic syndrome, which typically includes a finding of at least two, preferably three or more of the following symptoms: blood pressure equal to or higher than 130/85 mmHg; fasting blood sugar (glucose) equal to or higher than 100 mg/dL; large waist circumference (length around the waist): Men—40 inches or more, Women—35 inches or more; low HDL cholesterol: Men—under 40 mg/dL, Women—under 50 mg/dL, Triglycerides equal to or higher than 150 mg/dL.

In some embodiments, a method for treating or inhibiting the progression of a metabolic disorder or disease in a subject in need thereof by administering to the subject an effective amount of a disclosed composition. The subject can display one or more symptoms selected from the group consisting of excessive appetite relative to healthy subjects, elevated blood glucose levels relative to healthy subjects, increased glucose sensitivity relative to healthy subjects, increased glycosylated protein levels relative to healthy subjects, elevated insulin levels relative to healthy subjects, decreased insulin sensitivity relative to healthy subjects, increased blood triglyceride levels relative to healthy subjects, increased blood cholesterol levels relative to healthy subjects, increased blood free fatty acid levels relative to healthy subjects, or a combination thereof. The metabolic disorder or disease can be selected from the list consisting of prediabetes, impaired fasting glycaemia, impaired glucose tolerance (IGT), dysglycemia, insulin resistance, hypertriglyceridemia, hyperglyceridemia, stroke, arteriosclerotic vascular disease (ASVD), dyslipoproteinemia, hypolipoproteinemia, and hyperlipidemia or hypercholesterolemia.

Comorbidities of metabolic disorders include heart disease, vascular disease, atherosclerosis, diabetes, heart attack, kidney disease, nonalcoholic fatty liver disease, peripheral artery disease, and stroke.

In some embodiments, the disclosed compositions are used to prevent, improve, reduce, delay, or improve one or more symptoms or comorbidities of metabolic disorder.

Methods of treating a metabolic disorder can also include dietary modifications such as reduced fat, increased fruits, vegetables, and whole-grain products, increase fish or fish oils; increased exercise; weight loss; managing blood pressure and blood sugar; and not smoking. Combination therapies can include administration of the compositions disclosed herein in combination with a second therapeutic agent that is known in the art for treating insulin resistance, Type 1 or 2 diabetes mellitus, high cholesterol, high blood lipids, metabolic syndrome, or a symptom of comorbidity thereof. For example, the compositions can be administered in combination with insulin or a cholesterol-lowering drug. Cholesterol lowering drugs include, but are not limited to, statins such as atorvastatin (Lipitor), simvastatin (Zocor), lovastatin (Mevacor), pravastatin (Pravachol), and rosuvastatin (Crestor).

2. Weight Gain and Obesity

In some embodiments, the disclosed compositions are used to reduce or decrease, total body weight in a subject. The disclosed compositions can also be used to reduce, decrease, or delay a rise in total body weight over time. In some embodiments, the compositions disclosed herein delay a rise in total body weight, for example, that which can occur over time in subjects with high fat diets, little or no exercise, hereditary mutations, hormone changes, advancing age (i.e., elderly), diabetes, high cholesterol or high triglycerides.

The disclosed compositions and methods can be used for treating or preventing obesity or one or more symptoms or comorbidities thereof.

In some embodiments the subject is a healthy individual of normal weight, or is already overweight, and any additional weight gain could result in obesity or obesity-associate comorbidities. Body Mass Index is a standardized method of determining a subject's weight category using a calculus that is known in the art. A subject can be, for example, underweight: BMI of less 18.5; normal weight: BMI of 18.5-24.9; overweight: BMI of 25-29.9; or obese: BMI of 30 or greater. Therefore, in preferred embodiments, the disclosed compositions are useful for treating or preventing weight gain in a subject with a normal BMI, an overweight BMI, or an obese BMI. For example, the disclosed compositions can be used to treat or prevent weight gain in a subject with a BMI of about 25, 26, 27, 28, 29, 30, or more.

In some embodiments, the subject consumes less food, for example, 0.5%, 1%, 2%, 3%, 4%, 5%, 10%, 15%, 20%, 25%, or 50% less food, over time while being administered the compositions.

3. Endurance and Physical Activity

The disclosed compositions and methods are useful for increasing or improving physical activity; increasing or improving endurance; or combinations thereof compared to, for example, a matched, untreated subject. The terms “endurance” or “stamina” as used herein mean the ability or strength to continue or last, especially despite fatigue, stress, or other adverse conditions. Therefore, in some embodiments, a subject treated with the disclosed compositions continues or persists in a physical activity longer compared to a control. In some embodiments, a subject treated with disclosed compositions completes a physical activity faster compared to a control. Controls can include, for example, the subject prior to treatment, or an untreated subject. Physical activities include, but are not limited to, walking, jogging, running, biking, swimming, and lifting. Increased endurance can include an increase in the duration of the physical activity, or an increase in intensity of the physical activity over the same duration. In some embodiments, the disclosed compositions are used to reduce or delay a decline in endurance or physical activity over time, for example with increasing age or in an elderly subject.

In some embodiments, the disclosed compositions are used to reduce or decrease, fatigue in a subject. In some embodiments, the disclosed compositions are used to reduce, decrease, or delay fatigue in a subject over time. As used herein “fatigue” can also include weakness, exhaustion, lethargy, languidness, languor, lassitude and listlessness.

4. Muscle Loss

The disclosed compositions and methods are useful for reducing muscle loss.

Over time, aging individuals can experience one or more of the following: (1) muscle fibers are reduced in number and shrink in size, (2) muscle tissue is replaced more slowly and lost muscle tissue is replaced with a tough, fibrous tissue, (3) changes in the nervous system cause muscles to have reduced tone and ability to contract. These changes can contribute to fatigue, weakness and reduced tolerance to exercise. The disclosed compositions can used to reduce or delay (1), (2), (3), or combinations thereof. For example, in a preferred embodiment, the disclosed compositions reduce or delay loss of muscle, build-up of fibrous tissue, or combinations thereof. The formation of fibrous tissue such as collagen or cartilage can be around the muscle or bone of a subject. In some embodiments, the disclosed compositions prevent, delay, reduce, or inhibit loss of muscle in a subject over time. In some embodiments, the disclosed compositions reduce the ratio of fibrous tissue to muscle in a subject. In some embodiments, the disclosed compositions reduce an increase in the ratio of fibrous tissue to muscle in a subject over time, for example in aging individuals. The ratio of fibrous tissue:muscle can be a reduction in the fibrous:muscle ratio throughout the body of a subject, or in a discrete location, such as around joints. Fibrous tissue includes, but is not limited to collagen, cartilage, and combinations thereof.

In some embodiments, the subject suffers from a disease or condition such as muscle atrophy, muscular dystrophy, sarcopenia, frailty, or combinations thereof. The disclosed compositions and methods can be used to treat or prevent muscle atrophy, muscular dystrophy, sarcopenia, frailty, combinations thereof, or one or more symptoms or comorbidities thereof. The muscle atrophy or sarcopenia can result from cachexia, immobilization, aging, chronic disease, cancer, or combinations thereof.

Sarcopenia typically refers to the loss of skeletal muscle mass associated with advancing age (Cruz-Jentoft, A. et al., Age and Aging, 39:412-423 (2010); Lang, T. et al., Osteoporosis Int, 21:543-559 (2010)). Loss of skeletal muscle mass can also be unrelated to age. For example, loss of skeletal muscle mass occurs in subjects with cachexia.

Muscle is a dynamic tissue that responds to damage and loss throughout the entire life of an individual. For example, following a physiological stimuli such as exercise or injury the muscle tissue responses by mounting a regenerative response that restores the cytoarchitecture within about a two week period (Shi and Garry, et al., Genes Dev., 20(13):1692-708 (2006) citing Hawke and Garry, J. Appl. Physiol., 91:534-551 (2001); Cossu and Biressi, Semin. Cell Dev. Biol., 16:623-631 (2005); Dhawan and Rando, Trends Cell Biol., 15:666-673 (2005); Holterman and Rudnicki, Semin Cell Dev. Biol., 16:575-584 (2005)). It has been suggested that sacropenia results, at least in part, from the loss of muscle regenerative capacity. As used herein, muscle regenerative capacity can be affected by the rate of muscle regeneration, the rate of formation of fibrous tissue, or combinations thereof. For example, in some individuals, muscle regenerative capacity is low or reduced when muscle tissue is replaced more slowly and lost muscle tissue is replaced with a tough, fibrous tissue. Therefore, muscle regenerative capacity can be increased by increasing the rate of formation or amount of muscle mass, reducing the rate of formation or amount of fibrous tissue, or any combination thereof. In some embodiments, the disclosed compositions and methods are used to increase muscle regenerative capacity in a subject in need thereof. Certain embodiments provide methods for treating sacropenia in a subject by administering to the sacropenic subject an effective amount of one or more of the disclosed compositions to increase or promote muscle regenerative capacity in the subject. In some embodiments, an increase in muscle regenerative capacity is characterized by decreased or delay increase in fibrous tissue:muscle ratio.

Other factors that can contribute to the onset or progression of sacropenia include mitochondrial dysfunction (Lang, T. et al., Osteoporosis Int, 21:543-559 (2010)). The build-up of reactive oxygen species can cause mitochondrial dysfunction, which impairs muscle respiration and could contribute to muscle fiber deterioration. Thus, another embodiment provides a method for treating sacropenia in a subject by administering to the subject an effective amount of the disclosed compositions to reduce or inhibit mitochondrial dysfunction in the subject. Still another embodiment provides treating sarcopenia in subject in need thereof by administering to the subject an effective amount of the disclosed compositions to reduce or inhibit mitochondrial dysfunction in the subject.

Methods for measuring changes in skeletal muscle mass are known in the art. See for example Lang, T. et al., Osteoporosis Int, 21:543-559 (2010). Briefly, changes in skeletal muscle mass with age can be determined using lean body mass measurements with dual X-ray absorptiomety (DXA) and with muscle cross-sectional areas quantified by three-dimensional imaging methods such as X-ray computed tomography (CT) or with magnetic resonance imaging (MRI). Leg lean muscle mass by DXA can be used as a marker for skeletal muscle mass. Thus, changes in leg lean muscle mass can be used to detect or assisting in the diagnosis of sarcopenia or to monitor the effectiveness of a treatment for sacropenia.

In some embodiments, the subject has frailty syndrome. Frailty syndrome is a syndrome of multiple co-existing conditions including weakness, immobility, and poor tolerance to physiological and psychological stressors. (Sara Espinoza and Jeremy Walston, Cleveland Journal Clinical Medicine, 72(12):1105-1112 (2005)). Individuals diagnosed with frailty syndrome are referred to as “frail.” Frailty can exist in subjects regardless of age, disability, or disease.

5. Hypertension

The disclosed compositions and methods can be used to treat or prevent the development or progression of high blood pressure or hypertension. Primary hypertension has no known cause. The methods and compositions disclosed herein can be combined with other methods of treating and preventing of hypertension including maintaining normal body weight, reducing dietary sodium intake, engaging in regular aerobic physical activity such as brisk walking, limiting alcohol consumption, consuming a diet rich in fruit and vegetables, consuming a diet with reduced content of saturated and total fat, and combinations thereof.

The disclosed compositions may aid, augment, replace or supplement such lifestyle changes such as increased physical activity, maintain or achieve normal body weight, and lower blood lipids in a similar way a changed diet may.

Insulin resistance and obesity are known risk factors of hypertension. Disclosed compositions may reduce weight increases and insulin resistance and may hence reduce the risk of developing hypertension, or may reduce blood pressure in a patient with hypertension, insulin resistance, obesity, or combinations thereof.

C. Combination Therapy

In some embodiments, the methods include administration of one or more active agents that increase WAT beiging in combination with one or more additional active agents. The additional active agent can be, for example, a traditional therapy for the disease or disorder being treated or another compound that induced or increases WAT. Exemplary compounds are discussed in more detail below.

The term “combination” or “combined” is used to refer to either concomitant, simultaneous, or sequential administration of two or more active agent compounds. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

### III. Compositions

Compositions are also provided. The compositions include direct and indirect inhibitors of targets Brd9, Ankib1, Cacng1, and/or Gtl3 (Cfap20), and gene products thereof. Thus, in some embodiments, the inhibitors directly or indirectly reduce bioactivity, expression, location, activity, or a combination thereof the Brd9, Ankib1, Cacng1, and/or Gtl3 (Cfap20) gene, mRNA, protein, or a combination thereof. In some embodiments, the compound is an inhibitory polypeptide; a small molecule or peptidomimedic, or an inhibitory nucleic acid that targets genomic or expressed Brd9, Ankib1, Cacng1, and/or Gtl3 (Cfap20) nucleic acids, or a vector that encodes an inhibitory nucleic acid. The compound can reduce the expression or bioavailability of a gene product of Brd9, Ankib1, Cacng1, and/or Gtl3 (Cfap20). Inhibition can be competitive, non-competitive, uncompetitive, or product inhibition. Thus, an inhibitor can directly inhibit Brd9, Ankib1, Cacng1, and/or Gtl3 (Cfap20), can inhibit another factor in a pathway that leads to induction, persistence, or amplification of Brd9, Ankib1, Cacng1, and/or Gtl3 (Cfap20) expression, or a combination thereof.

Exemplary inhibitors are described below. The disclosed compounds also include tautomers, isomers, epimers, diastereoisomer, as well as any form of the compounds, such as the base (zwitter ion), pharmaceutically acceptable salts, e.g., pharmaceutically acceptable acid addition salts, hydrates or solvates of the base or salt, as well as anhydrates, and also amorphous, or crystalline forms thereof.

Pharmaceutical compositions including an effective amount of one or more inhibitors are also provided.

A. Target Sequences

Nucleic acid and amino acid sequences for Brd9, Ankib1, Cacng1, and Gtl3 (Cfap20) are known in the art.

1. Brd9

Bromodomain-containing protein 9 (BRD9) belongs to a family of bromodomain-containing proteins (including 46 proteins in human). Bromodomains are chromatin readers that recognize acetyl-/propionyl-/butyryl-modified lysine residues on histones (Brand, M., et al., ACS Chem Biol, 10, 22-39 (2015)).

Sequences for human Brd9 can be found at, for example, UniProtKB—Q9H8M2 (BRD9_HUMAN), which is specifically incorporated by reference herein in its entirety, and provides exemplary canonical, isoform 1, as well as numerous alternative isoforms and variants thereof.

A nucleic acid sequence encoding canonical isoform 1 is NCBI Reference Sequence: NM_023924.4, Homo sapiens bromodomain containing 9 (BRD9), transcript variant 1, mRNA, which is specifically incorporated by reference herein in its entirety.

Genomic sequences are also known in the art. See, for example, Gene ID:65980 (BRD9 bromodomain containing 9 [Homo sapiens (human)]), which provides Homo sapiens chromosome 5, GRCh38.p12 Primary Assembly NCBI Reference Sequence: NC_000005.10 (863735 . . . 892824, complement), each of which is specifically incorporated by reference herein.

2. Ankib1

Ankyrin repeat and IBR domain-containing protein 1 (Ankib1) may act as an E3 ubiquitin-protein ligase, or as part of E3 complex, which accepts ubiquitin from specific E2 ubiquitin-conjugating enzymes and then transfers it to substrates.

Sequences for human Ankib1 can be found at, for example, UniProtKB—Q9P2G1 (AKIB1_HUMAN), which is specifically incorporated by reference herein in its entirety, and provides an exemplary amino acid sequence, as well as several variants thereof.

A nucleic acid sequence encoding the amino acid sequence of Q9P2G1 is NCBI Reference Sequence: NM_019004.1, Homo sapiens ankyrin repeat and IBR domain containing 1 (ANKIB1), mRNA, which is specifically incorporated by reference herein in its entirety.

Genomic sequences are also known in the art. See, for example, Gene ID:54467 (ANKIB1 ankyrin repeat and IBR domain containing 1 [Homo sapiens (human)]), which provides Homo sapiens chromosome 7, GRCh38.p12 Primary Assembly, NCBI Reference Sequence: NC_000007.14 (92246223 . . . 92401773), each of which is specifically incorporated by reference herein.

3. Cacng1

Voltage-dependent calcium channel gamma-1 subunit (CACNG1) is a subunit of the dihydropyridine (DHP) sensitive calcium channel, and plays a role in excitation-contraction coupling. The skeletal muscle DHP-sensitive Ca2+ channel may function only as a multiple subunit complex.

Sequences for human Cacng1 can be found at, for example, UniProtKB—Q06432 (CCG1_HUMAN), which is specifically incorporated by reference herein in its entirety, and provides an exemplary amino acid sequence, as well as a variant thereof.

A nucleic acid sequence encoding the amino acid sequence of Q06432 is NCBI Reference Sequence: NM_000727.3, Homo sapiens calcium voltage-gated channel auxiliary subunit gamma 1 (CACNG1), mRNA, which is specifically incorporated by reference herein in its entirety.

Genomic sequences are also known in the art. See, for example, Gene ID:786 (CACNG1 calcium voltage-gated channel auxiliary subunit gamma 1 [Homo sapiens (human)]), which provides Homo sapiens chromosome 17, GRCh38.p12 Primary Assembly, NCBI Reference Sequence: NC_000017.11 (67044536 . . . 67056797), each of which is specifically incorporated by reference herein.

4. Gt13 (Cfap20)

GLT3, also referred to herein as, and can be used interchangeably with, Cilia- and flagella-associated protein 20 (CFAP20). The human gene and protein are typically referred to as Cfap20. GLT3 (CFAP20) is a cilium- and flagellum-specific protein that plays a role in axonemal structure organization and motility, and is involved in the regulation of the size and morphology of cilia.

Sequences for human Gtl3 (Cfap20) can be found at, for example, UniProtKB—Q9Y6A4 (CFA20_HUMAN), which is specifically incorporated by reference herein in its entirety, and provides an exemplary amino acid sequence, as well as a variant thereof.

A nucleic acid sequence encoding the amino acid sequence of Q9Y6A4 is NCBI Reference Sequence: NM_013242.2, Homo sapiens cilia and flagella associated protein 20 (CFAP20), mRNA, which is specifically incorporated by reference herein in its entirety.

Genomic sequences are also known in the art. See, for example, Gene ID:29105 (CFAP20 cilia and flagella associated protein 20 [Homo sapiens (human)]), which provides Homo sapiens chromosome 16, GRCh38.p12 Primary Assembly, NCBI Reference Sequence: NC_000016.10 (58113588 . . . 58129425, complement), each of which is specifically incorporated by reference herein.

B. Inhibitors

1. Pharmacological Inhibitors

In some embodiments, the inhibitor is a pharmacological inhibitor, for example a small molecule inhibitor. “Small molecule” as used herein, refers to an organic molecule, inorganic molecule, or organometallic molecule having a molecular weight less than 2000, 1500, 1200, 1000, 750, or 500 atomic mass units.

a. Inhibitors of Brd9

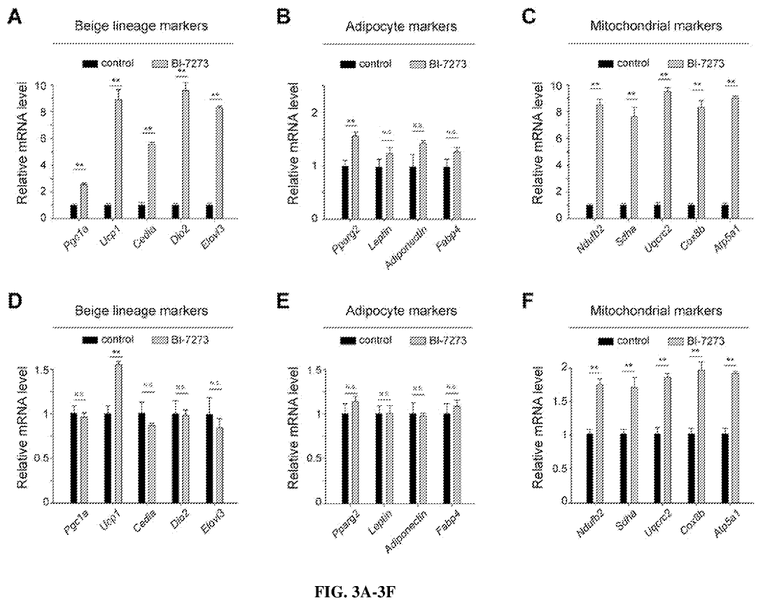

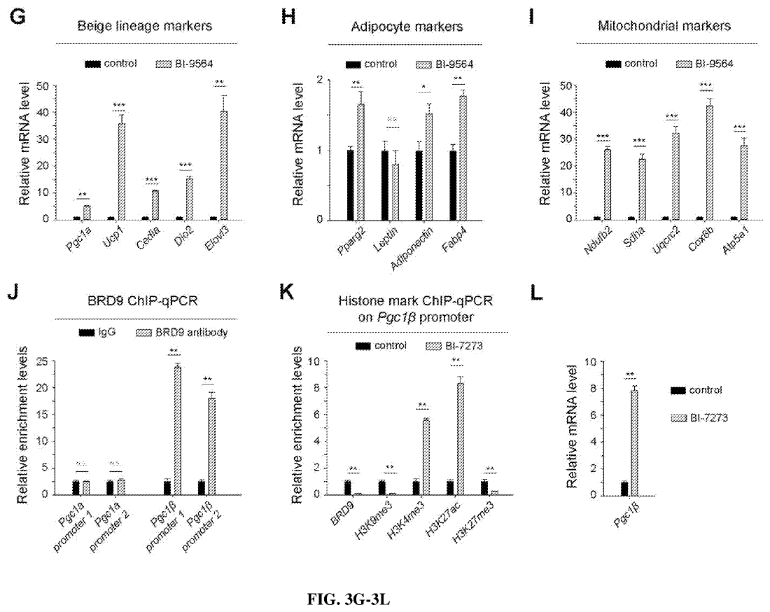

The experiments below show that inhibition of BRD9 can markedly reduced the enrichment of BRD9 on the Pgc1β promoter (FIG. 3H), which is highly consistent with the notion that some inhibitors can displace BRD9 from its genomic binding sites (Hohmann, A. F., et al., Nat Chem Biol, 12, 672-679 (2016)). As a result, H3K4me3, H3K27ac marks accumulated, whereas H3K9me3, H3K27me3 marks reduced on the Pgc1β proximal promoter (FIG. 3H), indicating that Brd9 epigenetically represses Pgc1β. In support of this, BI-7273 treatment indeed increased the Pgc1β mRNA level in proliferative white adipose progenitor cells (FIG. 3I). Therefore, these data indicate that BRD9 directly binds the proximal promoter region of Pgc1β gene in white adipose progenitor cells and epigenetically establishes a repressive chromatin environment, which leads to the repression of Pgc1β transcription and beige adipogenic lineage determination. Thus, in some embodiments, the BRD9 inhibitor epigenetically establishes a repressive chromatin environment by, for example, increasing H3K4me3, H3K27ac marks and decreasing H3K9me3, H3K27me3 marks on the Pgc1β proximal promoter.

The deep pocket of bromodomain is a prominent drug target for small molecules (Brand, M., et al., ACS Chem Biol, 10, 22-39 (2015); and Filippakopoulos, P., et al., FEBS Lett, 586, 2692-2704 (2012)). Non-selective inhibitors of bromodomain, such as JQ1 and I-BET, compete with the modified lysine residues and displace bromodomain-containing proteins from chromatin (Nicodeme, E., et al., Nature, 468, 1119-1123 (2010); and Filippakopoulos, P., et al., Nature, 468, 1067-1073 (2010)). These inhibitors display anti-tumor effects towards a variety of cancers, particularly hematopoietic malignancies (Suzuki, A., et al., Nucleic Acids Res, 42, 13557-13572 (2014), Muller, et al., Expert Rev. Mol. Med., 13, 1-21 (2011)). Other bromodomain inhibitors are known in the art. See, for example, U.S. Pat. No. 9,492,460.

Of all bromodomain-containing proteins, BRD9 is one of the only two chromatin readers that recognize butyryl-modified lysines (Flynn, E. M., et al., Structure, 23, 1801-1814 (2015)). Potent and selective inhibitors of BRD9 have been recently developed, and are discussed in more detail below.

LP99 was reported to be the first potent inhibitor of bromodomain-containing BRD7 and BRD9 (Clark, et al., “LP99: Discovery and Synthesis of the First Selective BRD7/9 Bromodomain Inhibitor,” Angew Chem Weinheim Bergstr Ger, 127, 6315-6319 (2015), which is specifically incorporated by reference herein in its entirety).

LP99 (IUPAC Name: N-[(2R,3S)-2-(4-chlorophenyl)-1-(1,4-dimethyl-2-oxoquinolin-7-yl)-6-oxopiperidin-3-yl]-2-methylpropane-1-sulfonamide) has the structure:

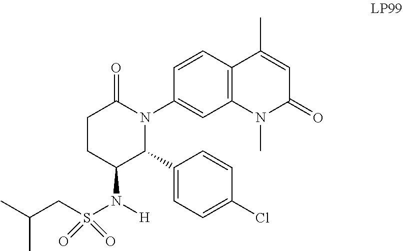 [SMILES: Cc1cc(=O)n(C)c2cc(N3C(=O)CC[C@H](NS(=O)(=O)CC(C)C)[C@H]3c3ccc(Cl)cc3)ccc12]

The first selective cellular chemical probe specific for BRD9 (I-BRD9; >700-fold selectivity over the BET family and 200-fold over the highly homologous BRD7) was developed through structure-based design (Theodoulou, et al., “Discovery of I-BRD9, a Selective Cell Active Chemical Probe for Bromodomain Containing Protein 9 Inhibition,” J Med Chem 59, 1425-1439 (2016), which is specifically incorporated by reference herein in its entirety).

I-BRD9 (IUPAC Name: N′-(1,1-dioxothian-4-yl)-5-ethyl-4-oxo-7-[3-(trifluoromethyl)phenyl]thieno[3,2-c]pyridine-2-carboximidamide), can have the structure:

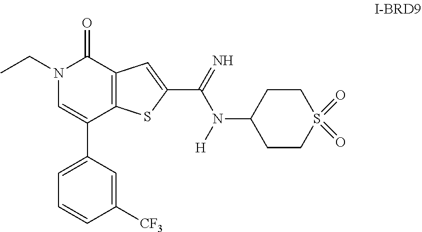 [SMILES: CCn1cc(-c2cccc(C(F)(F)F)c2)c2sc(C(=N)NC3CCS(=O)(=O)CC3)cc2c1=O]

or a resonance form shown below:

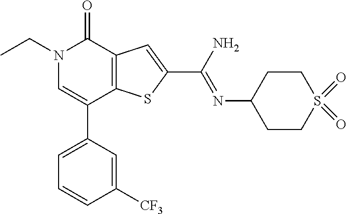 [SMILES: CCn1cc(-c2cccc(C(F)(F)F)c2)c2sc(/C(N)=N/C3CCS(=O)(=O)CC3)cc2c1=O]

Potent and selective BRD9 bromodomain inhibitors, BI-7273 and BI-9564, are reported to suppress the rapid cell proliferation and MYC expression in acute myeloid leukemia (AML) cells in vitro (Hohmann, et al., “Sensitivity and engineered resistance of myeloid leukemia cells to BRD9 inhibition,” Nat Chem Biol 12, 672-679 (2016), which is specifically incorporated by reference herein in its entirety) and show good bioavailability and antitumor activity in an AML xenograft model (Martin, et al., “Structure-Based Design of an in Vivo Active Selective BRD9 Inhibitor,” J Med Chem 59, 4462-4475 (2016), Karim and Schönbrunn, “An advanced tool to interrogate BRD9.” J. Med. Chem. 59(10), 4459-4461 (2016), which are specifically incorporated by reference herein in their entireties).

BI-7273 (IUPAC Name: 4-[4-[(dimethylamino)methyl]-3,5-dimethoxyphenyl]-2-methyl-2,7-naphthyridin-1-one), has the structure:

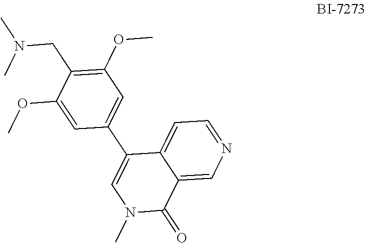 [SMILES: COc1cc(-c2cn(C)c(=O)c3cnccc23)cc(OC)c1CN(C)C]

BI-9564 (IUPAC Name: 4-[4-[(dimethylamino)methyl]-3,5-dimethoxyphenyl]-2-methyl-2,7-naphthyridin-1-one), has the structure:

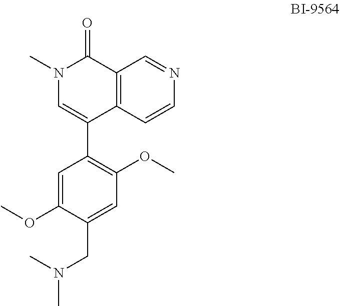 [SMILES: COc1cc(-c2cn(C)c(=O)c3cnccc23)c(OC)cc1CN(C)C]

Combinatorial treatment of BRD9 inhibitor (I-BRD9 or BI-9564) with cytostatic compounds (e.g., cisplatin) have been shown to synergistically inhibit the proliferation of rhabdoid tumor cells (Kramer, et al., “BRD9 Inhibition, Alone or in Combination with Cytostatic Compounds as a Therapeutic Approach in Rhabdoid Tumors,” Intl Mol Sci., 18(7). pii: E1537. doi: 10.3390/ijms18071537 (2018), which is specifically incorporated by reference herein in its entirety).

GNE-375, has been shown to prevent the drug resistance development in EGFR mutant PC9 cancer cells (Crawford, et al., “Inhibition of bromodomain-containing protein 9 for the prevention of epigenetically-defined drug resistance,” Bioorg Med Chem Lett 27, 3534-3541 (2017), which is specifically incorporated by reference herein in its entirety).

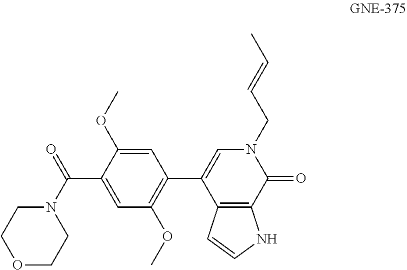 [SMILES: C/C=C/Cn1cc(-c2cc(OC)c(C(=O)N3CCOCC3)cc2OC)c2cc[nH]c2c1=O]

Notably, GNE-375 showed little effects on cell viability and global gene expression in this study but specifically decreases the expression of ALDH1A1—a previously confirmed beige adipocyte lineage repressor (Crawford, supra, Kiefer, et al., “Retinaldehyde dehydrogenase 1 regulates a thermogenic program in white adipose tissue,” Nat Med 18, 918-925 (2012)). Thus, BRD9 may repress beige lineage determination by multiple mechanisms (by inducing ALDH1A1 and repressing PGC1β expression), which is in concert with the robustness of BRD9 inhibitor in inducing WAT beiging.

In some embodiments, the compound is more effective, safer, exhibits fewer undesirable side effects, can used at lower dosage, can be administered less frequently, or a combination thereof compared to an alternative treatment. For example, recent studies demonstrated the potential of a couple of synthetic compounds with defined targets and mechanisms in inducing WAT beiging. Of these beiging compounds, rosiglitazone, lobeglitazone, and GQ-16 belong to a class of full/partial agonists for PPARγ (Coelho, M. S., et al., PLoS One, 11, e0154310 (2016); Jiang, C., et al., Mol Ther, 25, 1718-1729 (2017); and Merlin, J., et al., Cell Signal, 42, 54-66 (2018)). Thiazolidinediones (e.g., rosiglitazone, lobeglitazone) have common side-effects of weight gain, hyperphagia, edema, and anemia; whereas oral administration of GQ-16 (40 mg/kg/d, for 14 days) only induced a mediocre level of WAT beiging (5-fold increased of Ucp1). Roscovitine (Seliciclib, CYC202) is an inhibitor of cyclin-dependent kinases 2, 7, 9 (CDK2, CDK7, CDK9), which is currently in clinical trials for advanced solid tumors, Cushings diseases, and cystic fibrosis. Intraperitoneal injection of Roscovitine (50 mg/kg/d for 42 days) induced WAT beiging by blocking the phosphorylation of PPARγ (S275) by CDKs (Wang, H., et al., Cell Metab, 24, 835-847 (2016)). Transcriptome analysis revealed that Roscovitine-induced UCP1^{+pos} beige adipocytes resemble PPARγ agonists-induced beige adipocytes but differ from catecholamines- or synthetic sympathetic-induced beige adipocytes (Wang, H., et al., Cell Metab, 24, 835-847 (2016)). Known side-effects of Seliciclib include electrolyte disturbances, gastrointestinal disturbances, fatigue and transient hyperglycemia, and elevation of liver enzymes. PPARγ activation in muscle does not elicit an antidiabetic effect (Quinn, C. E., et al., Br J Pharmacol, 153, 636-645 (2008)).

Thus, compared to above PPARγ agonists, Brd9 inhibitors are advantageous regarding their potential in improving PGC1β-dependent mitochondrial biogenesis, fatty acid oxidation capacity and insulin sensitivity in skeletal muscle and many other tissues.

Besides PPARγ agonists, three other synthetic compounds have been shown to induce WAT beiging. ZD7155 is a potent and selective competitive antagonist for the angiotensin II type 1 (AT1) receptor with side effects of angioedema, hypotension and acute pancreatitis (Junggren, I. L., et al., J Pharm Pharmacol, 48, 829-833 (1996)). Intraperitoneal injection of ZD7155 (1 mg/kg/d for 14 days) only led to very mild WAT beiging (less than 2-fold increased of Ucp1) (Than, A., et al., Signal Transduct Target Ther, 2, 17022 (2017)). Dibenzazepine (YO-01027) is a dipeptidic γ-secretase inhibitor that inhibits Notch signaling (van Es, J. H., et al., Nature, 435, 959-963 (2005)). Intraperitoneal injection of Dibenzazepine (4.6 mg/kg/d every other day for 1 month) induced mild WAT beiging (3-fold increased of Ucp1)(73). Due to the off-target effects of Dibenzazepine, a recent study explored the use of Dibenzazepine-loaded poly(lactide-co-glycolide)(PEG) nanoparticles in WAT local injection and its efficacy in treating obesity in mouse models (Jiang, C., et al., Mol Ther, 25, 1718-1729 (2017)). Bexarotene is a synthetic retinoid that selectively activates retinoid X receptors and has been approved by FDA for treatment of cutaneous T cell lymphoma. Oral administration of Bexarotene (50 mg/kg/d, for 28 days) induced very mild WAT beiging only in subcutaneous WATs (1.5-fold increased of Ucp1) (Nie, B., et al., Cell Rep, 18, 624-635 (2017)). Compared to ZD7155, Dibenzazepine and Bexarotene, Brd9 inhibitor BI-7273 induced extensive WAT beiging and improvement in insulin sensitivity. As discussed above, the markable antidiabetic effect of BI-7273 was likely not only due to WAT beiging but also a result of the whole-body induction of PGC1β.

b. Inhibitors of Cacng1

The Cacng1 gene encodes the voltage-dependent calcium channel gamma-1 subunit. L-type calcium channels are composed of five subunits. The protein encoded by Cacng1 is one of several gamma subunit proteins. This particular gamma subunit is part of skeletal muscle 1,4-dihydropyridine-sensitive calcium channels and is an integral membrane protein that plays a role in excitation-contraction coupling. It is also a member of the neuronal calcium channel gamma subunit gene subfamily of the PMP-22/EMP/MP20 family and is located in a cluster with two similar gamma subunit-encoding genes.

Inhibitors of Cacng1 and gene products thereof are known in the art. For example, in some embodiments, the inhibitor is a calcium channel blocker (CCB) (also referred to as calcium channel antagonists or calcium antagonists). CCB's include several medications that disrupt the movement of calcium (Ca2+) through calcium channels. They have been used as antihypertensive drugs, i.e., as medications to decrease blood pressure in patients with hypertension.

In preferred embodiments, the CCB is a dihydropyridine. Dihydropyridine (DHP) calcium channel blockers are derived from the molecule dihydropyridine and often used to reduce systemic vascular resistance and arterial pressure. This CCB class is easily identified by the suffix “-dipine.”

Numerous DHP calcium channel blockers are known in the art, and several have been approved by the Food and Drug Administration. Exemplary, non-limiting DHP calcium channel blockers include, but are not limited to, amlodipine (NORVASC), aranidipine (SAPRESTA), azelnidipine (CALBLOCK), barnidipine (HYPOCA), benidipine (CONIEL), cilnidipine (ATELEC, CINALONG, SISCARD), clevidipine (CLEVIPREX), efonidipine (LANDEL), felodipine (PLENDIL), isradipine (DYNACIRC, PRESCAL), lacidipine (MOTENS, LACIPIL), lercanidipine (ZANIDIP), manidipine (CALSLOT, MADIPINE), nicardipine (CARDENE, CARDEN SR), nifedipine (PROCARDIA, ADALAT), nilvadipine (NIVADIL), nimodipine (NIMOTOP), nisoldipine (BAYMYCARD, SULAR, SYSCOR), nitrendipine (CARDIF, NITREPIN, BAYLOTENSIN), and pranidipine (ACALAS).

2. Functional Nucleic Acids Inhibitors

The inhibitor can be a functional nucleic acid. Functional nucleic acids are nucleic acid molecules that have a specific function, such as binding a target molecule or catalyzing a specific reaction. As discussed in more detail below, functional nucleic acid molecules can be divided into the following non-limiting categories: antisense molecules, siRNA, miRNA, aptamers, ribozymes, triplex forming molecules, RNAi, external guide sequences, and other gene editing compositions. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Therefore the compositions can include one or more functional nucleic acids designed to reduce expression of the Brd9, Ankib1, Cacng1, or Gtl3 (Cfap20) gene, or a gene product thereof. For example, the functional nucleic acid or polypeptide can be designed to target and reduce or inhibit expression or translation of Brd9, Ankib1, Cacng1, or Gtl3 (Cfap20) mRNA; or to reduce or inhibit expression, reduce activity, or increase degradation of Brd9, Ankib1, Cacng1, or Gtl3 (Cfap20) protein. In some embodiments, the composition includes a vector suitable for in vivo expression of the functional nucleic acid.

In some embodiments, a functional nucleic acid or polypeptide is designed to target a segment of the nucleic acid sequence encoding Brd9, Ankib1, Cacng1, or Gtl3 (Cfap20), or the complement thereof, or a genomic sequence corresponding therewith, or variants thereof having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a sequence encoding Brd9, Ankib1, Cacng1, and Gtl3 (Cfap20).

In some embodiments, a functional nucleic acid or polypeptide is designed to target a segment of a the nucleic acid encoding the amino acid sequence of Brd9, Ankib1, Cacng1, and Gtl3 (Cfap20), or the complement thereof, or variants thereof having a nucleic acid sequence 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a nucleic acid encoding the amino acid sequence of Brd9, Ankib1, Cacng1, and Gtl3 (Cfap20).

In some embodiments, the function nucleic acid hybridizes to the nucleic acid encoding Brd9, Ankib1, Cacng1, and Gtl3 (Cfap20), or a complement thereof, for example, under stringent conditions. In some embodiments, the functional nucleic acid hybridizes to a nucleic acid sequence that encodes the amino acid sequence of Brd9, Ankib1, Cacng1, and Gtl3 (Cfap20), or a complement thereof, for example, under stringent conditions.

a. Antisense

The functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

b. Aptamers

The functional nucleic acids can be aptamers. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹°, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the K_{d} with a background binding molecule. It is preferred when doing the comparison for a molecule such as a polypeptide, that the background molecule be a different polypeptide.

c. Ribozymes

The functional nucleic acids can be ribozymes. Ribozymes are nucleic acid molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

d. Triplex Forming Oligonucleotides

The functional nucleic acids can be triplex forming molecules. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹°, or 10⁻¹².

e. External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Representative examples of how to make and use EGS molecules to facilitate cleavage of a variety of different target molecules are known in the art.

f. RNA Interference

In some embodiments, the functional nucleic acids induce gene silencing through RNA interference. Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III—like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs.

Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors having shRNA are available, such as, for example, Imgenex's GENESUPPRESSOR™ Construction Kits and Invitrogen's BLOCK-IT™ inducible RNAi plasmid and lentivirus vectors.

In some embodiment, the functional nucleic acid is siRNA, shRNA, miRNA. In some embodiments, the composition includes a vector expressing the functional nucleic acid. Methods of making and using vectors for in vivo expression of functional nucleic acids such as antisense oligonucleotides, siRNA, shRNA, miRNA, EGSs, ribozymes, and aptamers are known in the art.

g. Other Gene Editing Compositions

In some embodiments the functional nucleic acids are gene editing compositions. Gene editing compositions can include nucleic acids that encode an element or elements that induce a single or a double strand break in the target cell's genome, and optionally a polynucleotide. The compositions can be used, for example, to reduce or otherwise modify expression of Brd9, Ankib1, Cacng1, or Gtl3 (Cfap20).

h. Strand Break Inducing Elements CRISPR/Cas

In some embodiments, the element that induces a single or a double strand break in the target cell's genome is a CRISPR/Cas system. CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with the required elements including a Cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the ‘target sequence’ and the tracrRNA is often referred to as the ‘scaffold’.

In some embodiments, one or more elements of a CRISPR system is derived from a type I, type II, or type III CRISPR system. In some embodiments, one or more elements of a CRISPR system is derived from a particular organism including an endogenous CRISPR system, such as Streptococcus pyogenes.

In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence can be any polynucleotide, such as DNA or RNA polynucleotides. In some embodiments, a target sequence is located in the nucleus or cytoplasm of a cell.

In the target nucleic acid, each protospacer is associated with a protospacer adjacent motif (PAM) whose recognition is specific to individual CRISPR systems. In the Streptococcus pyogenes CRISPR/Cas system, the PAM is the nucleotide sequence NGG. In the Streptococcus thermophiles CRISPR/Cas system, the PAM is the nucleotide sequence is NNAGAAW. The tracrRNA duplex directs Cas to the DNA target consisting of the protospacer and the requisite PAM via heteroduplex formation between the spacer region of the crRNA and the protospacer DNA.

Typically, in the context of an endogenous CRISPR system, formation of a CRISPR complex (including a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. All or a portion of the tracr sequence may also form part of a CRISPR complex, such as by hybridization to all or a portion of a tracr mate sequence that is operably linked to the guide sequence.

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequences.

In some embodiments, one or more vectors driving expression of one or more elements of a CRISPR system are introduced into a target cell such that expression of the elements of the CRISPR system direct formation of a CRISPR complex at one or more target sites.

For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element can be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector includes one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector includes an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector includes two or more insertion sites, each insertion site being located between two tracr mate sequences so as to allow insertion of a guide sequence at each site. In such an arrangement, the two or more guide sequences can include two or more copies of a single guide sequence, two or more different guide sequences, or combinations of these. When multiple different guide sequences are used, a single expression construct may be used to target CRISPR activity to multiple different, corresponding target sequences within a cell. For example, a single vector can include about or more than about 1, 2, 3, 4, 5, 6, 7, 8, 9, 10, 15, 20 guide sequences. In some embodiments, about or more than about 1, 2, 3, 4, 5, 6, 7, 8, 9, 10, such guide-sequence-containing vectors may be provided, and optionally delivered to a cell.

In some embodiments, a vector includes a regulatory element operably linked to an enzyme-coding sequence encoding a CRISPR enzyme, such as a Cas protein.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, a vector encodes a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III) can be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%>, 1%>, 0.1%>, 0.01%, or lower with respect to its non-mutated form.

In some embodiments, an enzyme coding sequence encoding a CRISPR enzyme is codon optimized for expression in particular cells, such as eukaryotic cells. The eukaryotic cells can be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules.

The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al., Nucl. Acids Res., 28:292 (2000). Computer algorithms for codon optimizing a particular sequence for expression in a particular host cell, for example Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid.

In some embodiments, a vector encodes a CRISPR enzyme including one or more nuclear localization sequences (NLSs). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

In general, the one or more NLSs are of sufficient strength to drive accumulation of the CRISPR enzyme in a detectable amount in the nucleus of a eukaryotic cell. In general, strength of nuclear localization activity may derive from the number of NLSs in the CRISPR enzyme, the particular NLS(s) used, or a combination of these factors.

Detection of accumulation in the nucleus may be performed by any suitable technique. For example, a detectable marker may be fused to the CRISPR enzyme, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g., assay for DNA cleavage or mutation at the target sequence, or assay for altered gene expression activity affected by CRISPR complex formation and/or CRISPR enzyme activity), as compared to a control no exposed to the CRISPR enzyme or complex, or exposed to a CRISPR enzyme lacking the one or more NLSs.

In some embodiments, one or more of the elements of CRISPR system are under the control of an inducible promoter, which can include inducible Cas, such as Cas9.

Cong, Science, 15:339(6121):819-823 (2013) reported heterologous expression of Cas9, tracrRNA, pre-crRNA (or Cas9 and sgRNA) can achieve targeted cleavage of mammalian chromosomes. Therefore, CRISPR system utilized in the methods disclosed herein can be encoded within a vector system which can include one or more vectors which can include a first regulatory element operably linked to a CRISPR/Cas system chimeric RNA (chiRNA) polynucleotide sequence, wherein the polynucleotide sequence includes (a) a guide sequence capable of hybridizing to a target sequence in a eukaryotic cell, (b) a tracr mate sequence, and (c) a tracr sequence; and a second regulatory element operably linked to an enzyme-coding sequence encoding a CRISPR enzyme which can optionally include at least one or more nuclear localization sequences. Elements (a), (b) and (c) can arranged in a 5′ to 3 orientation, wherein components I and II are located on the same or different vectors of the system, wherein when transcribed, the tracr mate sequence hybridizes to the tracr sequence and the guide sequence directs sequence-specific binding of a CRISPR complex to the target sequence, and wherein the CRISPR complex can include the CRISPR enzyme complexed with (1) the guide sequence that is hybridized to the target sequence, and (2) the tracr mate sequence that is hybridized to the tracr sequence, wherein the enzyme coding sequence encoding the CRISPR enzyme further encodes a heterologous functional domain. In some embodiment, one or more of the vectors encodes also encodes a suitable Cas enzyme, for example, Cas9. The different genetic elements can be under the control of the same or different promoters.

While the specifics can be varied in different engineered CRISPR systems, the overall methodology is similar. A practitioner interested in using CRISPR technology to target a DNA sequence (such as Brd9, Ankib1, Cacng1, and Gtl3 (Cfap20)) can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

Exemplary, non-limiting sgRNA sequence can target human Brd9 include Brd9:

sgRNA1:
(SEQ ID NO: 53)
5′ CACCGAGATACCGTGTACTACAAGT 3′
sgRNA2:
(SEQ ID NO: 54)
5′ CACCGAGGGAGCACTGTGACACGGA 3′

Zinc Finger Nucleases

In some embodiments, the element that induces a single or a double strand break in the target cell's genome is a nucleic acid construct or constructs encoding a zinc finger nucleases (ZFNs). ZFNs are typically fusion proteins that include a DNA-binding domain derived from a zinc-finger protein linked to a cleavage domain.

The most common cleavage domain is the Type IIS enzyme Fold. Fok1 catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31,978-31,982 (1994b). One or more of these enzymes (or enzymatically functional fragments thereof) can be used as a source of cleavage domains.

The DNA-binding domain, which can, in principle, be designed to target any genomic location of interest, can be a tandem array of Cys₂His₂ zinc fingers, each of which generally recognizes three to four nucleotides in the target DNA sequence. The Cys₂His₂ domain has a general structure: Phe (sometimes Tyr)-Cys-(2 to 4 amino acids)-Cys-(3 amino acids)-Phe(sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

Engineering methods include, but are not limited to, rational design and various types of empirical selection methods. Rational design includes, for example, using databases including triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

Transcription Activator-Like Effector Nucleases

In some embodiments, the element that induces a single or a double strand break in the target cell's genome is a nucleic acid construct or constructs encoding a transcription activator-like effector nuclease (TALEN). TALENs have an overall architecture similar to that of ZFNs, with the main difference that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically they differ principally at two positions (amino acids 12 and 13, called the repeat variable diresidue, or RVD). Each RVD specifies preferential binding to one of the four possible nucleotides, meaning that each TALEN repeat binds to a single base pair, though the NN RVD is known to bind adenines in addition to guanine. TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011). US Published Application No. 2011/0145940, which discloses TAL effectors and methods of using them to modify DNA. Miller et al. Nature Biotechnol 29: 143 (2011) reported making TALENs for site-specific nuclease architecture by linking TAL truncation variants to the catalytic domain of Fold nuclease. The resulting TALENs were shown to induce gene modification in immortalized human cells. General design principles for TALE binding domains can be found in, for example, WO 2011/072246.

i. Gene Altering Polynucleotides

The nuclease activity of the genome editing systems described herein cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site.

Therefore, in some embodiments, the genome editing composition optionally includes a donor polynucleotide. The modifications of the target DNA due to NHEJ and/or homology-directed repair can be used to induce gene correction, gene replacement, gene tagging, transgene insertion, nucleotide deletion, gene disruption, gene mutation, etc.

Accordingly, cleavage of DNA by the genome editing composition can be used to delete nucleic acid material from a target DNA sequence by cleaving the target DNA sequence and allowing the cell to repair the sequence in the absence of an exogenously provided donor polynucleotide. Thus, the subject methods can be used to knock out a gene (resulting in complete lack of transcription or altered transcription) or to knock in genetic material into a locus of choice in the target DNA.

Alternatively, if the genome editing composition includes a donor polynucleotide sequence that includes at least a segment with homology to the target DNA sequence, the methods can be used to add, i.e., insert or replace, nucleic acid material to a target DNA sequence (e.g., to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, the compositions can be used to modify DNA in a site-specific, i.e., “targeted”, way, for example gene knock-out, gene knock-in, gene editing, gene tagging, etc. as used in, for example, gene therapy.

In applications in which it is desirable to insert a polynucleotide sequence into a target DNA sequence, a polynucleotide including a donor sequence to be inserted is also provided to the cell. By a “donor sequence” or “donor polynucleotide” or “donor oligonucleotide” it is meant a nucleic acid sequence to be inserted at the cleavage site. The donor polynucleotide typically contains sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence includes a non-homologous sequence flanked by two regions of homology, such that homology-directed repair between the target DNA region and the two flanking sequences results in insertion of the non-homologous sequence at the target region.

Donor sequences can also include a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence can include certain sequence differences as compared to the genomic sequence, e.g., restriction sites, nucleotide polymorphisms, selectable markers (e.g., drug resistance genes, fluorescent proteins, enzymes etc.), etc., which can be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence can be a single-stranded DNA, single-stranded RNA, double-stranded DNA, or double-stranded RNA. It can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. Proc. Natl. Acad. Sci. USA 84:4959-4963 (1987); Nehls et al. Science 272:886-889 (1996). Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues.

As an alternative to protecting the termini of a linear donor sequence, additional lengths of sequence can be included outside of the regions of homology that can be degraded without impacting recombination. A donor sequence can be introduced into a cell as part of a vector molecule having additional sequences such as, for example, replication origins, promoters and genes encoding antibiotic resistance.

C. Delivery Vehicles

The disclose compounds can be administered and taken up into the cells of a subject with or without the aid of a delivery vehicle. Appropriate delivery vehicles for the disclosed inhibitors are known in the art and can be selected to suit the particular inhibitor. For example, if the compound is a nucleic acid or vector, the delivery vehicle can be a viral vector, for example a commercially available preparation, such as an adenovirus vector (Quantum Biotechnologies, Inc. (Laval, Quebec, Canada). The viral vector delivery can be via a viral system, such as a retroviral vector system which can package a recombinant retroviral genome (see e.g., Pastan et al., (1988) Proc. Natl. Acad. Sci. U.S.A. 85:4486; Miller et al., (1986) Mol. Cell. Biol. 6:2895). The recombinant retrovirus can then be used to infect and thereby deliver to the infected cells nucleic acid encoding the compound inhibitor. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948 (1994)), adeno-associated viral (AAV) vectors (Goodman et al., Blood 84:1492-1500 (1994)), lentiviral vectors (Naidini et al., Science 272:263-267 (1996)), pseudotyped retroviral vectors (Agrawal et al., Exper. Hematol. 24:738-747 (1996)).

Physical transduction techniques can also be used, such as liposome delivery and receptor-mediated and other endocytosis mechanisms (see, for example, Schwartzenberger et al., Blood 87:472-478 (1996)). For example in some embodiments, the CTPS1 inhibitor is delivered via a liposome. Commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art are well known. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.). This disclosed compositions and methods can be used in conjunction with any of these or other commonly used gene transfer methods.

In some embodiments, the delivery vehicle is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the compound. In some embodiments, release of the drug(s) is controlled by diffusion of the compound out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

D. Formulations

The disclosed compounds can be formulated in a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

The compositions can be administered systemically.

Drugs can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are typically prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et. al., (Media, Pa.: Williams and Wilkins, 1995) which provides information on carriers, materials, equipment and process for preparing tablets and capsules and delayed release dosage forms of tablets, capsules, and granules.

The compound can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by, or bound to, a nanoparticle, microparticle, micelle, synthetic lipoprotein particle, or carbon nanotube. For example, the compositions can be incorporated into a vehicle such as polymeric particles which provide controlled release of the active agent(s). In some embodiments, release of the drug(s) is controlled by diffusion of the active agent(s) out of the particles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing particles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

1. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Oral Immediate Release Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

3. Extended release dosage forms

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form having single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is used in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

4. Delayed release dosage forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, Pa.: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

5. Formulations for Mucosal and Pulmonary Administration

Active agent(s) and compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions composed of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

6. Topical and Transdermal Formulations

Transdermal formulations may also be prepared. These will typically be gels, ointments, lotions, sprays, or patches, all of which can be prepared using standard technology. Transdermal formulations can include penetration enhancers.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) composed of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

### EXAMPLES

The following abbreviations are used:

iWAT: inguinal white adipose tissue

eWAT: epididymal white adipose tissue

iBAT: interscapular brown adipose tissue

SVF: stromal vascular fraction

PGC1α: peroxisome proliferator-activated receptor γ co-activator 1α

PGC1β: peroxisome proliferator-activated receptor γ co-activator 1β

UCP1: uncoupling protein-1

Cidea: Cell Death-Inducing DFFA-Like Effector A

Dio2: Iodothyronine Deiodinase 2

Elovl3: ELOVL Fatty Acid Elongase 3

Cox8b: Cytochrome C Oxidase Subunit 8B

NST: non-shivering thermogenesis

### Example 1: A Primary Screen Identifies sgRNAs that are Effective to Induce Beige Adipocyte Formation In Vitro

### Materials and Methods

Animals

All animal studies were approved by the University of Georgia Institutional Animal Care and Use Committee (IACUC) and performed strictly following the guidelines Animals were housed in a temperature-controlled (22° C.) environment with 12:12-hrs light:dark cycle and allowed food and water ad lithium. All mouse strains were purchased from the Jackson Laboratory: C57BL/6J: #000664; Ucp1-Cre: #024670; ROSA-INTACT: #021039. To generate diet-induced obesity and type 2 diabetes models, 8-week old C57BL/6J mice (both males and females) were fed with a high-fat diet (60% fat, Research Diet, catelog #D12492) for 2 weeks. To perform gavage feeding, animals were immobilized by hands and a gavage solution (0.5% Natrosol or 0.5% Natrosol containing BI-7273, 150 μL) was administered through a 1 mL syringe equipped with a disposable gavage needle.

Isolation, Culture and Differentiation of Primary White Adipose Progenitor Cells

The stromal vascular fractions (SVFs) of iWAT and eWAT were isolated based on an established protocol (Aune, U. L., et al., J Vis Exp (2013)). Briefly, iWAT or eWAT was dissected, washed in PBS, minced into 3 mm³ small pieces, and digested with 1.5 mg/mL type II collagenase (Worthington, Lakewood, N.J., USA) in an isolation buffer (123 mM NaCl, 5 mM KCl, 1.3 mM CaCl₂, 5 mM glucose, 100 mM HEPES, and 4% fatty-acid-free BSA) for 45 min at 37° C. with frequent agitation. Tissue suspension was sequentially filtered through 100 and 70 μm cell strainers and centrifuged at 800×g for 5 min to pellet SVF cells.

The cell pellet was resuspended in a growth medium (DMEM high glucose containing 20% FBS and 1% penicillin/streptomycin), plated on collagen-coated cell culture plates, and cultured at 37° C. with 5% CO₂. To expand adipose progenitor cells, proliferative adipose progenitor cells were passaged for two times when the confluence reaches 70%.

To differentiate into adipocytes, white adipose progenitor cells were cultured in growth media to reach 100% confluence for 1 day. The growth media was changed into basal induction medium (DMEM high glucose containing 10% FBS, 0.5 mM isobutylmethylxanthine, 125 mM indomethacin, 2 μg/mL dexamethasone, 850 nM insulin, 1% penicillin/streptomycin). After 2-day induction, the media was changed to basal differentiation medium (DMEM high glucose containing 10% FBS, 850 nM insulin, 1% penicillin/streptomycin) and further incubated for 4-5 days. To differentiate beige adipocytes, fully confluent white adipose progenitor cells were incubated with the above induction medium supplemented with 2 mM rosiglitazone and 1 nM T3 for 2 days and further differentiated in the basal differentiation medium supplemented with 1 mM rosiglitazone and 1 nM T3 for 4-5 days.

High-Throughput Sequencing and Bioinformatic Analysis of sgRNA Sequences

Genomic DNA from FACS-isolated UCP1^{+pos} beige adipocytes were isolated by a Genomic DNA Isolation kit (Thermo Fisher Scientific). Genomic DNA was separated into 20 PCR tubes and a 20-bp region containing sgRNA sequences were PCR amplified by the following primers:

F2:
(SEQ ID NO: 1)
5′-AATGATACGGCGACCACCGAGATCTACACTCTTTCC
CTACACGACGCTCTTCCGATCTtcttgtggaaaggacga
aacaccg-3′

For the variable region, no base is in primer F2a (used for pooled plasmid libraries), TTT is in primer F2b (used for iWAT library) (SEQ ID NO:55) and AAAAAA is in primer F2c (used for eWAT library) (SEQ ID NO:56).

R2B1:
(SEQ ID NO : 2)
5′-CAAGCAGAAGACGGCATACGAGATACATTGGCG
TGACTGGAGTTCAGACGTGTGCTCTTCCGATCTtct
actattattcccctgcactgt-3′

Amplified PCR products (260 bp) were purified by Zymo DNA Clean & Concentrator 20 kit and eluted into 20 uL. A 5% fraction of the PCR products pooled from 20 PCR tubes were used in a second round of PCR amplification to add adaptor sequences and variable barcodes to the amplifed gsRNA sequences. The products (360 bp) from the second round of PCR were pooled and submitted to sequencing on an Illumina HiSeq2500 sequencer (Hudson Alpha). ˜25 million reads were sequenced. An in-house developed bioinformatic pipeline were used to analyze the sequencing reads. Briefly, sequences from different biological samples were separated based on the variable regions in F2 primers. Sequences of each sample were mapped to the reference sgRNA sequences of GECKO v2 library by BWA. Mapping results were parsed by a perl program to count sequencing reads to individual sgRNAs. Candidate sgRNAs for secondary screening were determined by the following criteria: 1) the abundance of the sgRNA sequences in a biological sample was significantly (p<0.01) higher than that of the sequenced reference library; 2) more than one sgRNAs targeting the same gene were present in a biological sample (either iWAT or eWAT); 3) same sgRNA were present in both iWAT and eWAT samples. 14 sgRNA sequences met the 1) & 2) or 1) & 3) criteria and were synthesized as DNA oligos for secondary screening.

### Results

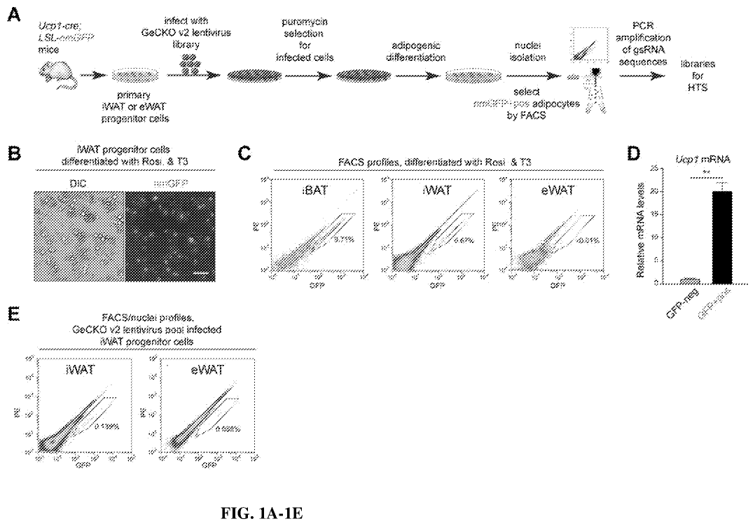

In this study, an sgRNA-based screening approach was used to identify beige adipocyte lineage repressors (FIG. 1A). To this aim, Ucp1-Cre;ROSA-LSL-nmGFP mice were generated, within which the Ucp1-Cre allele drives the expression of Cre recombinase in Ucp1^{+pos} cells, and the Cre recombinase, in turn, induces the permanent expression of nuclear membrane-localized GFP (nmGFP) from the LSL-nmGFP allele. As such, Ucp1^{+pos} brown/beige adipocytes can be specifically selected from a mixed cell culture based on their unique nmGFP expression. Adipogenic progenitor cells were generated from interscapular BAT depots (iBAT), inguinal WAT depots (iWAT), and epidydimal WAT depots (eWAT) in 5-week old Ucp1-Cre;ROSA-LSL-nmGFP mice. These WAT adipogenic progenitor cells were nmGFP^{-neg}, consistent with the notion that UCP1 is expressed during the lineage determination of brown/beige adipocytes but not in adipose progenitor cells.

To test if nmGFP expression can be induced in vitro, the adipogenic progenitor cells that were isolated from iWAT were differentiated under a condition that favors the beige adipocyte lineage determination—with basal adipogenic differentiation media supplemented with Rosiglitazone (Rosi.) and T3 thyroid hormone (T3; FIG. 1B). After five days of differentiation, adipocytes carrying multiple oil-droplets emerged in the culture (FIG. 1B, DIC); many of these adipocytes also had GFP^{+pos} nuclear membrane, confirming the inducible expression of nmGFP in vitro. Next, the iBAT, iWAT and eWAT progenitor cells were differentiated under the same conditions and the percentages of nmGFP^{+pos} adipocytes were analyzed in the cultures by fluorescence assisted cell sorting (FACS). FACS revealed that nmGFP^{+pos} brown/beige adipocytes counted for 9.7%, 0.67%, and less than 0.01% of iBAT, iWAT and eWAT cultures, respectively (FIG. 1C). These percentages are highly correlated with the brown/beige differentiation potential of these types of progenitor cells. UCP1 is a definitive marker of thermogenic beige adipocytes.

To confirm nmGFP^{+pos} cells were indeed beige adipocytes, RT-qPCR was performed to measure relative expression levels of Ucp1 mRNA in FACS-sorted nmGFP^{+pos} and nmGFP^{-neg} cells from the iWAT culture. nmGFP^{+pos} cells expressed ˜20-fold higher Ucp1 mRNA than nmGFP^{-neg} cells (FIG. 1D), validating the robustness of the above screening strategy in the identification of beige adipocytes in a mixed culture.

Next, iWAT and eWAT progenitor cells were infected with GeCKO v2 lentivirus pool. A multiplicity of infection (MOI) of 0.8 was achieved for both types of cells, which lowered the possibility of false identification of sgRNAs. After library infection, progenitor cells were cultured in puromycin-containing growth media for seven days to 1) select lentivirus-infected progenitor cells and 2) allow the occurrence of sgRNA-mediated genome editing. The Mouse GeCKO v2 library includes 130,209 sgRNAs, which are designed mostly targeting the first and second coding exons of protein-coding genes as well as functionally important regions of non-coding RNAs (Sanjana, N. E., et al., Nat Methods 11, 783-784). Multiple sgRNAs targeting the same gene (redundancy) are included in the GeCKO v2 library to minimize the bias associated with the variance of sgRNA-targeting efficiency.

After 7-day culture in growth media, ˜5×10⁷ puromycin-selected iWAT or eWAT progenitor cells were differentiated under a basal adipogenic differentiation media (without Rosi. or T3). This differentiation condition was chosen to identify those sgRNAs that are self-sufficient to drive beige adipogenic lineage determination without the aid of pro-beiging compounds. After 7-days of adipogenic differentiation, nuclei were isolated from cultured adipocytes, and nmGFP^{+pos} beige adipocyte nuclei were sorted by FACS. nmGFP^{+pos} beige adipocyte nuclei counted for 0.139% and 0.058% of total nuclei in cultured derived from iWAT and eWAT progenitor cells, respectively (FIG. 1E). Compared to the culture differentiated under the pro-beiging condition (FIG. 1C), nmGFP^{+pos} beige adipocytes counted for a lower percentage in GeCKO-infected iWAT culture that were differentiated under the basal adipogenic differentiation media (0.67% vs. 0.139%; FIG. 1E). The lower percentage is consistent with the notion that adipose progenitors from iWAT are prone to differentiate into beige adipocytes in vitro in the presence of pro-beiging compounds (Aune, U. L., et al., J Vis Exp (2013)). Intriguingly, the increased percentage of beige adipocytes in GeCKO-infected eWAT culture (<0.01% vs. 0.058%) indicates that some beige lineage repressors were indeed targeted and ablated by sgRNAs in GeCKO libraries.

Next, genomic DNA was isolated from FACS-isolated beige adipocyte nuclei (nuclei from iWAT and eWAT cultures were pooled separately) and the sgRNA sequence-containing region was amplified by PCR. Nested PCR reactions were later performed to add sequence barcodes and adaptors at both ends of sgRNA PCR products. The sgRNA libraries (iWAT, eWAT, and original pooled plasmids in GeCKO libraries) were subjected to high-throughput sequencing (HTS) on a Solexa HiSeq-2500 sequencer. Using an in-house generated Perl script, the sgRNA sequences in the sequenced libraries were extracted and the sgRNA sequences were mapped back to their unique gene targets. HTS confirmed that 112,512 (86.4%) sgRNAs were included in the actual screening in this study. In the iWAT library, 1,441 unique sgRNAs were identified with a median sequencing depth of 14. In the eWAT library, 425 unique sgRNAs were identified with a median sequencing depth of 17. To determine a small number of most promising sgRNAs for the secondary screening, bioinformatic analysis was performed according to the following criteria: 1) the candidate sgRNA (pool A) had a higher abundance (sequencing depth) in either iWAT or eWAT library comparing to the pool plasmid libraries with a probability less than 0.05; 2) the candidate sgRNA (from pool A) was identified in both iWAT and eWAT libraries; 3) the candidate sgRNA from pool A targets a gene that had more than one sgRNAs in pool A. Using these stringent selection criteria, 14 candidate sgRNAs that target 10 candidate genes (beige lineage repressors) were identified.

### Example 2: A Secondary Screen Confirms the Efficacies of sgRNAs in Inducing Beige Adipocyte Formation from White Adipose Progenitor Cells

### Materials and Methods

H/E Staining and Immunohistochemistry (IHC)

For histology, iWAT was excised from mice and fixed with 10% formalin, dehydrated and embedded in paraffin. The sliced tissue (5 μm) was stained with hematoxylin and eosin Y (Sigma Aldrich). Cell size was quantified by NIH ImageJ (ver 1.47t). Alternative sections were used for immunohistochemistry detection of UCP1 and H/E staining. Paraffin sections were antigen unmasked with Tris-EDTA buffer (10 mM Tris Base, 1 mM EDTA, 0.05% Tween 20, pH 9.0) in 93° C. for 15 min. Sections were blocked with blocking solution (10% normal goat serum and 1% BSA in PBS) for 1 hr at room temperature and incubated with the primary antibody (anti-UCP1, 1:200 diluted in blocking solution) overnight at 4° C. The endogenous peroxidase was quenched with 3% hydrogen peroxide. Sections were incubated with the secondary antibody for 1 hr at room temperature. Slides were developed with DAB (VectorLab) for 5 min followed with hemotoxylin conterstaining for 1 min. Color images were captured on a Leica DM4000B microscope equipped with a DFC450c camera.

Quantitative Real-Time PCR

Total RNA was isolated from tissue or cell culture using TRIzol. Total RNA (500 ng) from tissues or cell culture was used in 25 μL reverse transcription reactions (Maxima; Life technologies) and diluted into 250 μL. Diluted cDNA (2 μL) was used in quantitative PCR reactions (SsoAdvanced™ Universal SYBR Green mix; Bio-Rad) on a BioRad CFX384™ Real-Time PCR Detection System. Cq-values were determined and expression values were calculated by BioRad CFX Manager™ Software. Relative mRNA expression values were normalized to inner reference genes Rps18 and Tbp of each biological sample. For mitochondrial copy number qPCR, mitochondrial Cox2 gene was amplified and normalized to genomic β-globin gene.

The following primers were used in RT-qPCR:

| | Sense | Anti-sense |
|---|---|---|
| | sequence | Sequence |
| Gene | 5′-3′ | 5′-3′ |
| Tbp | ACTTCGTGCA | TCTGGATTGTT |
| | AGAAATGCTG | CTTCACTCTTG |
| | A | G |
| | (SEQ ID | (SEQ ID |
| | NO: 3) | NO: 22) |
| Rps18 | CGCCATGTCT | GGTCGATGTCT |
| | CTAGTGATCC | GCTTTCCTC |
| | (SEQ ID | (SEQ ID |
| | NO: 4) | NO: 23) |
| Pparg2 | GCATGGTGCC | TGGCATCTCTG |
| | TTCGCTGA | TGTCAACCATG |
| | (SEQ ID | (SEQ ID |
| | NO: 5) | NO: 24) |
| Adipoq | AACTTGTGCA | TTCTCCAGGCT |
| | GGTTGGATGG | CTCCTTTCC |
| | (SEQ ID | (SEQ ID |
| | NO: 6) | NO: 25) |
| Fabp4 | CTGGGCGTGG | ACACATTCCAC |
| | AATTCGATGA | CACCAGCTT |
| | (SEQ ID | (SEQ ID |
| | NO: 7) | NO: 26) |
| Lep | CCAGGATGAC | CAGCACATTTT |
| | ACCAAAACCC | GGGAAGGCA |
| | (SEQ ID | (SEQ ID |
| | NO: 8) | NO: 27) |
| Pgc1b | TCCTGTAAAA | GCTCTGGTAGG |
| | GCCCGGAGTA | GGCAGTGA |
| | T (SEQ ID | (SEQ ID |
| | NO: 9) | NO: 28) |
| Cidea | ATACATCCAG | ACTTACTACCC |
| | CTCGCCCTTT | GGTGTCCAT |
| | (SEQ ID | (SEQ ID |
| | NO: 10) | NO: 29) |
| Cox8b | GAACCATGA | GCGAAGTTCAC |
| | AGCCAACGA | AGTGGTTCC |
| | CT | (SEQ ID |
| | (SEQ ID | NO: 30) |
| | NO: 11) | |
| Pgc1a | GGTCAAGAT | TCATAGCTGTC |
| | CAAGGTCCC | GTACCTGGG |
| | CA | (SEQ ID |
| | (SEQ ID | NO: 31) |
| | NO: 12) | |
| Ucp1 | ATACTGGCA | CGAGTCGCAGA |
| | GATGACGTC | AAAGAAGCC |
| | CC | (SEQ ID |
| | (SEQ ID | NO: 32) |
| | NO: 13) | |
| Dio2 | CAGTGTGGT | TGAACCAAAGT |
| | GCACGTCTC | TGACCACCAG |
| | (SEQ ID | (SEQ ID |
| | NO: 14) | NO: 33) |
| Elov13 | TCCGCGTTC | GGACCTGATGC |
| | TCATGTAGG | AACCCTATGA |
| | TCT | (SEQ ID |
| | (SEQ ID | NO: 34) |
| | NO: 15) | |
| Ndufb2 | CGATTTTGG | ATGTCTCACCT |
| | CATGACTCG | TGCTCCTACT |
| | GA | (SEQ ID |
| | (SEQ ID | NO: 35) |
| | NO: 16) | |
| Sdha | GCATTACAA | TTATCTCCAGG |
| | CATGGGTGG | CCTGCAAGA |
| | GA | (SEQ ID |
| | (SEQ ID | NO: 36) |
| | NO: 17) | |
| Uqcrc2 | GACCCCATC | TTGGTAAACTC |
| | TTGCTTTGC | AAGATCCTGAG |
| | T | G |
| | (SEQ ID | (SEQ ID |
| | NO: 18) | NO: 37) |
| Atp5a1 | AGACAGACT | GGAGTAGGGAG |
| | GGGAAAACA | CCAAGTACT |
| | TCG | (SEQ ID |
| | (SEQ ID | NO: 38) |
| | NO: 19) | |
| MitoCox2 | GCCGACTAA | CAATGGGCATA |
| | ATCAAGCAA | AAGCTATGG |
| | CA | (SEQ ID |
| | (SEQ ID | NO: 39) |
| | NO: 20) | |
| β-globin | GAAGCGAT | GGAGCAGCGAT |
| | TCTAGGGA | TCTGAGTAGA |
| | GCAG | (SEQ ID |
| | (SEQ ID | NO: 40) |
| | NO: 21) | |

In Vivo Knockout of Candidate Genes by Lentivirus Expressing Cas9/sgRNA

pLentiCRISPRv2 plasmids that express sgRNA targeting confirmed candidate genes (Brd9, Ankib1, Cacng1, Cfap20) from secondary screening were used to package lentiviruses by above-described PET transfection method. Before the injection, subject C57BL/6 mice were anesthetized with 1% isoflurane and the fur at the lower abdominal area were removed. For iWAT injection, small (˜3 mm) incisions were made on skin on top of inguinal WAT depots to expose the fat depots. About 1×10⁹ lentivirus particles were resuspended in 50 uL saline supplemented with 8 ug/mL Polybrene and injected into each iWAT depot. For eWAT injection, large incisions (˜10 mm) were sequentially made on skin and the muscle layer beneath the skin along the midline of lower abdomen to expose eWAT fat depots. About 1×10⁹ lentivirus particles were resuspended in 100 uL saline supplemented with 8 ug/mL Polybrene and injected into each eWAT depot at 2-3 sites. After injections, subject mice were sutured and received analgesic Ketoprofen. iWAT and eWAT samples were collected at 14 days post lentivirus injection.

### Results

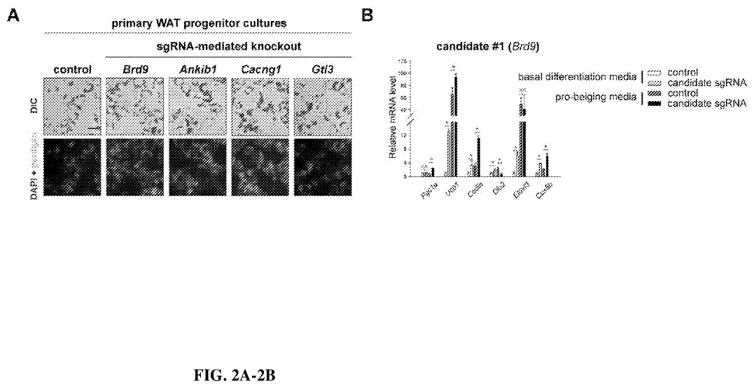

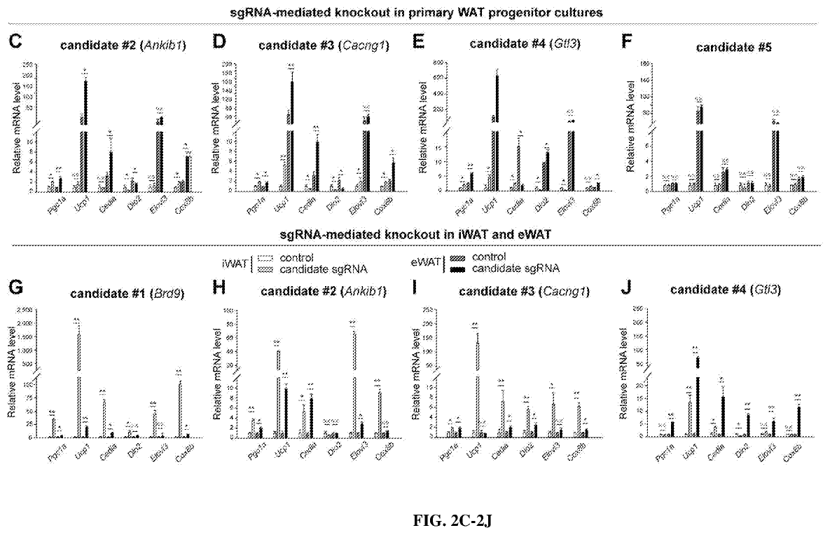

Next, oligos for the candidate sgRNAs were synthesized and cloned into lentiviral constructs (pLenti-Crispr-v2) that express these individual sgRNAs and the Cas9 nuclease. Lentiviruses packaged from these 14 candidate sgRNA constructs were used individually to infect primary adipose progenitor cells that were isolated from iWAT. Lentivirus-infected progenitor cells were selected in puromycin-containing media for 7 days and differentiated under both the basal adipogenic differentiation media (without Rosi. or T3) and pro-beiging differentiation media (with Rosi. and T3). RT-qPCR was performed to measure relative mRNA levels of beige adipocyte markers (Pgc1α, Ucp1, Cedia, Dio2, Elovl3, Cox8b), which were compared to the levels in control cells (infected with lentiviruses without a specific sgRNA sequence). Of 10 candidate genes, 4 genes were confirmed to be beige lineage repressors (Brd9, Ankib1, Cacng1, Gtl3). sgRNA-mediate knockout (KO) of these genes did not affect the adipogenic differentiation of white adipose progenitor cells as evidenced by the comparable numbers of Perilipin^{+pos} multiocular adipocytes in the differentiation cultures (FIG. 2A). The mRNA levels of thermogenic adipocyte definitive marker Ucp1 had ˜12.5-fold, 1.5-fold, 5.7-fold and 6.3-fold increases (compared to the control) under the basal adipogenic differentiation condition upon Brd9, Ankib1, Cacng1 and Gtl3 KO, respectively (FIG. 2B-2E). The mRNA levels of Pgc1α, a mitochondrial biogenesis master regulator, increased upon Ankib1, Cacng1 and Gtl3 KO (but not Brd9 KO; FIG. 2B-2E). The mRNA levels of Cox8b, a mitochondrial content marker, increased upon Brd9, Ankib1, and Cacng1 KO (but not Gtl3 KO; FIG. 2B-2E). Consistently, Brd9 KO adipocytes had increased expression levels of Cidea, Dio2, and Elovl3 under the basal adipogenic differentiation condition, indicating the emergence of thermogenic beige adipocytes in the culture. Under the pro-beiging differentiation condition, Brd9, Ankib1, Cacng1 and Gtl3 KO also increased the expression of beige adipocyte markers in variable levels (FIG. 2B-2E). In contrast, sgRNA KO of a non-beige lineage determinant did not affect the expression levels of the above beige adipocyte markers under either basal or pro-beiging differentiation condition (FIG. 2F, candidate #5). The above results indicate that individual knockout of Brd9, Ankib1, Cacng1, Gtl3 is sufficient to induce beige adipocyte formation in vitro from cultured white adipose progenitor cells without the aid of pro-beiging compounds.

The efficacies of the sgRNAs that target Brd9, Ankib1, Cacng1, and Gtl3 genes on inducing beige adipocyte formation were also tested in vivo. Lentiviruses packaged from the above sgRNA constructs or a non-specific control sgRNA were used individually to infect iWAT or eWAT of C57BL/6 mice. After 2 weeks, beige adipocytes were formed in iWAT that were infected with Brd9, Ankib1, Cacng1, and Gtl3 KO lentiviruses, which is indicated by the remarkable increases of beige adipocyte markers (Pgc1α, Ucp1, Cedia, Dio2, Elovl3, Cox8b) in the iWAT depots (FIG. 2G-2J). Brd9, Ankib1, Cacng1, and Gtl3 KO led to ˜1,500-fold, 42.5-fold, 131-fold and 13.2-fold increases of Ucp1 mRNA in iWAT, respectively, indicating extensive levels of beige adipocytes formation in vivo. In addition to iWAT, sgRNA-mediated Ankib1 and Gtl3 KO also induced beige adipocytes formation in eWAT, as evidenced by the increases of beige adipocyte markers (9.7-fold and 62-fold increases of Ucp1 mRNA; FIG. 2H, 2J). Compared to Ankib1 and Gtl3 KO, Brd9 and Cacng1 KO only induced mediocre increases of beige adipocyte markers in eWAT (FIG. 2G, 2I). Thus, Brd9 and Cacng1 KO may be more efficacious towards inducing iWAT beiging. The above data indicate that individual knockout of Brd9, Ankib1, Cacng1, Gtl3 is efficacious to induce beige adipocyte formation in vivo.

### Example 3: BI-7273 Induces Beige Adipogenic Lineage Determination and Epigenetically Activated Pgc1β

### Materials and Methods

Secondary Screening to Confirm the Efficacies of Candidate sgRNAs

SgRNA oligos (1 mol) were annealed in TE buffer on a PCR machine. Annealled oligos were phosphorylated at 5′ ends by T4 PNK and purified by Zymo DNA Clean & Concentrator 5 kit. Annealled sgRNA oligos were cloned into the BsmBI site of pLentiCRISPRv2 plasmids. Sequence-confirmed pLentiCRISPRv2-sgRNA plasmids were transfected into 293T cells by PEI. After 48 h of transfection, 293T culture media (DMEM containing 10% FBS and 1% penicillin/streptomycin) were collected, filtered with 0.45 μm filters and incubate with 10% Speedy Lentivirus Concentrator (Abm Inc.) for overnight at 4° C. On the next day, lentivirus-containing media were centrifuged at 4250×g for 30 min at 4° C. Pelleted lentiviruses were resuspended in cell culture media with 8 ug/mL Polybrene (Santa Cruz) and incubated with white adipose progenitor cells for 12 hrs. After 12-hr incubation, cells were washed 3 times in warm PBS and cultured in growth media for additional 48 hrs. After 48 hrs, infected cells were selected in growth media supplemented with puromycin (1 μg/mL) for additional 5 days. Selected cells were differentiated in adipogenic differentiation media (either basal or pro-beiging).

Chromatin Immunoprecipitation (ChIP) and ChIP-qPCR

Primary iWAT progenitor cells were culture in growth media and treated with BI-7273 (10 μM) for 48 hrs and reached 100% confluence. 1×10⁷ cells were fixed in 1% paraformaldehyde for 10 min at room temperature, washed with PBS and quenched with PBS supplemented with 125 mM glycine for 10 min Fixed cells were scratched from petri dishes, collected by centrifugation at 1000×g for 5 min, and resuspended in 100 μl lysis buffer (1% SDS, 10 mM EDTA, 50 mM Tris-HCl pH 8.1, lx protease inhibitor cocktail) and sonicated at 4° C. in a Bioruptor® Pico sonication device (Diagenode) using 5 on/off cycles of 30:30 seconds for 3 rounds (15 on/off sonication cycles in total to reach the average chromatin size ˜300 bp). Between each sonication round, sample tubes were vortexed and spinned briefly. The sheared chromatin was 1:10 diluted in IP dilution buffer (50 mM HEPES-KOH pH7.5, 140 mM NaCl, 1 mM EDTA, 1% Triton X-100, 0.1% sodium deoxycholate, 0.1% SDS) and centrifuged at 20,000×g, 4° C. for 10 min. The supernatant was transferred to siliconized tubes and incubated with antibodies: 5 μg BRD9 antibody (rabbit, Active Motif #61537) or 5 ug histone H3K27ac antibody (rabbit, Active Motif #39133) or 5 ug histone H3K27me3 antibody (rabbit, Active Motif #39155) or 5 ug histone H3K9me3 antibody (rabbit, Active Motif #39161) or 5 ug histone H3K4me3 antibody (rabbit, Active Motif #39915) or 5 μg rabbit normal IgG (Santa Cruz) on a rotating platform at 4° C. overnight. On the next day, PBS-washed protein A Dynabeads (from 30 μl 50% slurry) were added to the chromatin/antibody mix and incubated on a rotating platform at 4° C. for 4 hrs. All ChIP beads were sequentially washed with low salt, high salt, lithium and TE buffer for 2 times/each and reverse-crosslinked with 1M NaCl for 6 hrs at 65° C. Decrosslinked samples were sequentially treated with 10 μg RNase A (Thermo Fisher Scientific) at 37° C. for 0.5 hr and 20 μg Proteinase K (Thermo Fisher Scientific) at 55° C. overnight. ChIPed genomic DNA was purified by phenol/chloroform/isoamyl alcohol (25:24:1) extraction followed by precipitation with 100% isopropanol and glycol blue (Thermo Fisher Scientific) at −20° C. overnight. The following primers were used to quantify relative enrichment levels at proximal promoter regions of Pgc1α and Pgc1β genes.

Pgc1α_promoter1_S:
(SEQ ID NO: 41)
5′-CTATTAAAAAGTAGGCTGGG-3′
Pgc1α_promoter1_AS:
(SEQ ID NO: 42)
5′-GCTGGCTTCAGTCACAGTGTG-3′
Pgc1α_promoter2_S:
(SEQ ID NO: 43)
5′-CCCTAGACTCTTGGGTTC-3′
Pgc1α_promoter2_AS:
(SEQ ID NO: 44)
5′-GGCACCCTGAAGCCATGAG-3′
Pgc1β_promoter 1_S:
(SEQ ID NO: 45)
5′-AGCGAGCAAGCGAGCGAGGA-3′
Pgc1β_promoterl_AS:
(SEQ ID NO: 46)
5′-GAGACCTGGTCCCTGCGC-3′
Pgc1β_promoter2_S:
(SEQ ID NO: 47)
5′-GCTCCGGCAGCCAGGTG-3′
Pgc1β_promoter2_AS:
(SEQ ID NO: 48)
5′-GCCTCAGTTTCCCCAGCTGTG-3′

The following primers were designed for PCR amplification of gene-lacking regions on chromosome 5 and 6 of mouse genome, which serve as inner reference controls in calculation of relative enrichment levels:

Chr5_S:
(SEQ ID NO: 49)
5′-CCCGTCACTCAACCATTTCA-3′
Chr5_AS:
(SEQ ID NO: 50)
5′-CTTATCAATGGGGGCTCTGG-3′
Chr6_S:
(SEQ ID NO: 51)
5′-AGATATGGCTGGCTTTGTGC-3′
Chr6_AS:
(SEQ ID NO: 52)
5′-GAACTCGCTCAGGTTCTGC-3′

### Results

Of the confirmed beige lineage repressors, BRD9 represents a promising therapeutic target for obesity and type 2 diabetes since 1) the bioinformatic analysis indicated that the expression level of BRD9 is significantly higher in omental adipose tissues of obese prepubertal children who are prediposed to insulin resistance and metabolic syndrome comparing to control lean children (GEO profile: GSE9624)(Aguilera, C. M., et al., Int J Mol Sci, 16, 7723-7737 (2015)); 2) the data from the secondary screening indicated that Brd9 KO induced beige adipocyte formation from white adipose progenitor cells under a basal adipogenic differentiation condition (FIG. 2B); and 3) recent studies have discovered potent and specific inhibitors for BRD9 (Hohmann, A. F., et al., Nat Chem Biol, 12, 672-679 (2016); Martin, L. J., et al., J Med Chem, 59, 4462-4475 (2016); and Theodoulou, N. H., et al., J Med Chem, 59, 1425-1439 (2016)-27)). The following experiments focus on the characterization of the therapeutic potential of a BRD9 inhibitor, BI-7273, which has been shown to have good bioavailability and in vivo activity (Martin, L. J., et al., J Med Chem, 59, 4462-4475 (2016)).

The efficacy of BI-7273 was tested by inducing beige adipocyte formation in vitro. Proliferative primary white adipose progenitor cells in growth media were treated with BI-7273 (10 μM) or 0.1% DMSO as a control for 48 hours before switching to a basal adipogenic differentiation condition (without further BI-7273 treatment). RT-qPCR indicated that BI-7273 treatment drastically increased the mRNA levels of beige lineage markers (Pgc1α, Ucp1, Cedia, Dio2, Elovl3) in the cultured compared to the DMSO control (FIG. 3A). In contrast, adipocyte markers (Pparg2, Leptin, Adiponectin, Fabp4) were not affected by BI-7273 treatment (FIG. 3B). Consistent with the induction of beige adipocytes in the culture, BI-7273 treatment also increased the mRNA levels of mitochondrial markers (Ndufb2, Sdha, Uqcrc2, Cox8b, Atp5a1; FIG. 3C). These data indicate that BI-7273 is a potent inducer of beige adipocyte formation from white adipose progenitor cells in vitro.

In the above lentivirus-based primary/secondary screenings, sgRNA-mediated genome editing and consequent gene KO could occur either in proliferative adipose progenitor cells or fully-differentiated adipocytes. As such, Brd9 may function as a beige lineage repressor by either repressing the beige adipocyte lineage differentiation or the white-to-beige adipocyte conversion. To distinguish the above possibilities, white adipocytes were differentiated from white adipose progenitor cells under the basal differentiation condition and the differentiated white adipocytes were treated with BI-7273 for 48 hours. In contrast to BI-7273 treatment of proliferative progenitor cells, BI-7273 treatment of differentiated white adipocytes only slightly increased Ucp1 mRNA for 1.6 fold and did not raise other beige lineage markers (Pgc1α, Cedia, Dio2, Elovl3; FIG. 3D). Adipogenic markers (Pparg2, Leptin, Adiponectin, Fabp4) were not affected by BI-7273 treatment of white adipocytes (FIG. 3E). Similar to the slight increase of Ucp1 mRNA, mitochondrial markers (Ndufb2, Sdha, Uqcrc2, Cox8b, Atp5a1) were also slightly increased from 1.7-2.0 fold in BI-7273 treated white adipocytes (FIG. 3F). These data indicate that BI-7273 is not sufficient to drive the white-to-beige adipocyte conversion in vitro. Therefore, BI-7273 likely induces the beige lineage determination and differentiation of white adipose progenitor cells by inhibiting BRD9.

BI-9564 is a newly developed tool compound that specifically inhibits BRD9 (KD=14 nM) but not other BET family members. To confirm that the inhibition of BRD9 (rather than that of BRD7) leads to beige adipogenic lineage determination, the efficacy of BI-9564 in inducing beige adipocyte and mitochondrial markers was tested in above-described primary white adipose progenitor cell cultures. Treatment with BI-9564 (and the control BI-6354) was carried out in the cultured adipocytes in the same manner as described for parallel assays with BI-7273.

RT-qPCR indicated that BI-9564 treatment markedly increased the mRNA levels of beige lineage markers (Pgc1α, Ucp1, Cedia, Dio2, Elovl3) and mitochondrial markers (Ndufb2, Sdha, Uqcrc2, Cox8b, Atp5a1) in the adipocyte culture compared to a negative control compound (BI-6354; FIG. 3G, 3I). In addition, adipocyte markers (Pparg2, Leptin, Adiponectin, Fabp4) were also induced by BI-9564 treatment at different levels (FIG. 3H). Thus, BRD9 is a potent inducer of beige adipogenic lineage for white adipose progenitor cells in vitro.

Next, the molecular mechanism by which BRD9 represses the beige adipogenic lineage determination was investigated. A recent study revealed that PGC1β, but not PGC1α, has an obligatory role in beige adipocyte lineage determination (Pardo, R., et al., PLoS One, 6, e26989 (2011)). To test whether BRD9 directly binds Pgc1β promoter, BRD9 chromatin immunoprecipitation (ChIP) was performed in proliferative white adipose progenitor cells using a BRD9 antibody. ChIP-qPCR indicated that BRD9 antibody enriched two proximal promoter regions of Pgc1β for 23.5 and 18 folds, indicating the direct association of BRD9 on the Pgc1β promoter (FIG. 3J). No enrichment was detected in two proximal promoter regions of Pgc1α (FIG. 3J). To reveal the function of BRD9 on the Pgc1β promoter, proliferative white adipose progenitor cells were treated with BI-7273 and ChIP was performed for BRD9 as well as histone marks that are associated with actively transcribed genes (H3K4me3, H3K27ac) and repressed genes (H3K9me3, H3K27me3) (Ruthenburg, A. J., et al., Nat Rev Mol Cell Biol, 8, 983-994 (2007)). ChIP-qPCR indicated that BI-7273 treatment markedly reduced the enrichment of BRD9 on the Pgc1β promoter (FIG. 3K), which is highly consistent with the notion that BI-7273 displaces BRD9 from its genomic binding sites (Hohmann, A. F., et al., Nat Chem Biol, 12, 672-679 (2016)). As a result, H3K4me3, H3K27ac marks accumulated, whereas H3K9me3, H3K27me3 marks reduced on the Pgc1β proximal promoter (FIG. 3K), indicating that BRD9 epigenetically represses Pgc1β. In support of this, BI-7273 treatment indeed increased the Pgc1β mRNA level in proliferative white adipose progenitor cells (FIG. 3L). Therefore, these data indicate that BRD9 directly binds the proximal promoter region of Pgc1β gene in white adipose progenitor cells and epigenetically establishes a repressive chromatin environment, which leads to the repression of Pgc1β transcription and beige adipogenic lineage determination.

Whole-transcriptome (RNA-seq) analysis of inguinal white fat (iWAT) was performed on cells isolated from mice fed with Brd9 inhibitor (or the control carrier). Analyses indicates that: 1) beige adipocyte markers were increased after Brd9 inhibitor treatment (confirming RT-qPCR data); 2) gene ontology analysis indicates Brd9 inhibitor treatment drastically increased the mitochondrial content in the white fat tissue; 3) inhibition of Brd9 reduced the expression level of Cacng1 (another beiging target from the screening), indicating these two candidates belong to the same genetic pathway.

### Example 4: Oral Administration of BI-7273 Induces Beiging in Both Subcutaneous and Visceral WATs

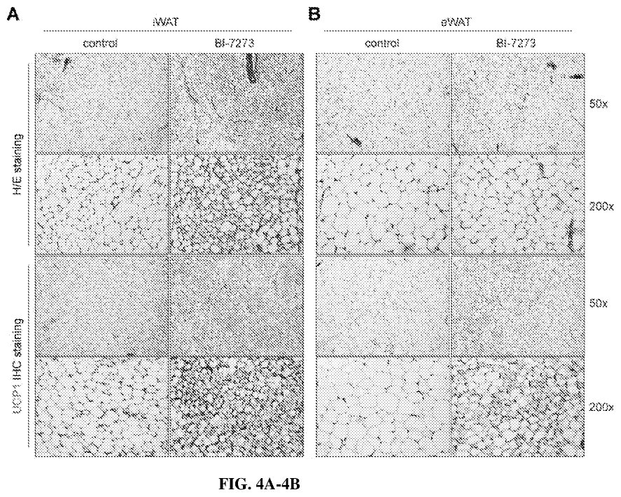

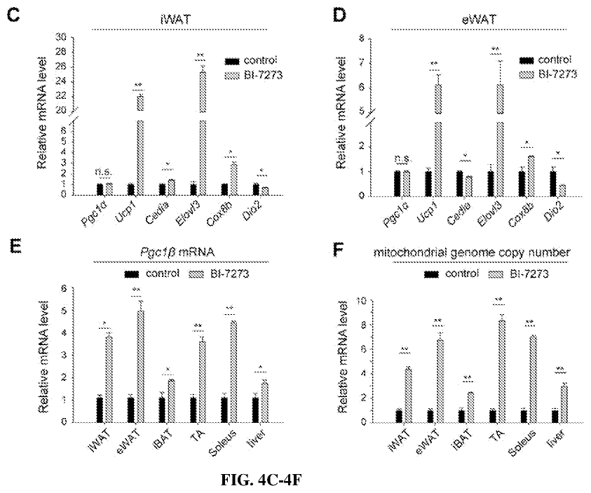

A previous study demonstrated the high bioavailability and in vivo activity of BI-7273 in an AML xenograft model (Martin, L. J., et al., J Med Chem, 59, 4462-4475 (2016)). The efficacy of BI-7273 in inducing beige adipocyte formation in vivo was tested within diet-induced obesity (DIO) and type 2 diabetes mouse models. To generate DIO models, adult C57BL/6 mice (8-week age, both males and females) were fed with a high-fat diet (HFD; 60% calories from fat) for 12 weeks. To administer BI-7273, BI-7273 mixed in 0.5% Natrosol (100 mg/kg/day, 200 uL) was gavaged for 14 days. For the control mice, 0.5% Natrosol in the same volume was gavaged during the same period. The high-fat diet was changed to the normal chow during gavaging to maximize the uptake of BI-7273. DIO models of control and BI-7273 treatment groups were euthanized after 14-day of gavaging and adipose tissues (iWAT and eWAT) were subjected to H/E staining and UCP1 immunohistochemistry (IHC) to identify beige adipocytes. For both iWAT and eWAT, BI-7273 overtly reduced the overall size of adipocytes as well as induced the formation of multiocular UCP1^{+pos} beige adipocytes (FIG. 4A, 4B). Compared to the control mice, RT-qPCR revealed that the mRNA levels of Ucp1 had a 22-fold and a 6-fold increase in iWAT and eWAT, respectively (FIG. 4C, 4D). Other beige adipocyte markers (Cedia, Dio2, Elovl3, Cox8b) were also increased by BI-7273 treatment (FIG. 4C, 4D). Notably, Pgc1α mRNA levels were not affected by BI-7273 treatment in either iWAT or eWAT (FIG. 4C, 4D). Intriguingly, a recent study showed that PGC1β can support the mitochondrial biogenesis during BAT lineage determination in the absence of PGC1a (Uldry, M., et al., Cell Metab, 3, 333-341 (2006)).

The effect of BI-7273 treatment on Pgc1β expression was further investigated. RT-qPCR indicated that the mRNA levels of Pgc1β had 3.8-fold and 4.9-fold increases in iWAT and eWAT after BI-7273 treatment, respectively (FIG. 4E). BI-7273 treatment also increased Pgc1β expression in iBAT, limb muscles [tibialis anterior (TA), extensor digitorum longus (EDL)], and liver (FIG. 4E). In line with increased Pgc1β expression, PCR confirmed that the mitochondrial genome copy number in the above tissues from BI-7273 treated DIO mice were 2.5-8.5 folds higher than those from control DIO mice (FIG. 4F). Therefore, BRD9 inhibitor, BI-7273, was efficacious to induce beige adipocyte formation within both iWAT and eWAT of DIO models. BI-7273 oral administration also elevated Pgc1β expression and mitochondrial biogenesis in multiple types of tissues.

### Example 5: Oral Administration of BI-7273 Augments Energy Expenditure and Reduced Body Weight and Adiposity

### Materials and Methods

Measurement of Whole-Body Energy Expenditure by Indirect Calorimetry

Indirect calorimetry was performed on a Columbus Instruments CLAMS system equipped with 8 metabolic cages. Oxygen consumption (VO₂; ml/min) and carbon dioxide production (VCO₂; ml/min) were measured at 22° C. with 12-hr:12-hr dark:light cycles. Total energy expenditure was estimated using the equation published before tusk 1928). Data were normalized to the body weight of each animal. Physical activity is measured by counting infrared beam breaking events.

Treadmill Exhaustion Test

Treadmill exhaustion test was performed with an Exer 3/6 treadmill and controller (Columbus Instruments). The treadmill was set up with a uphill inclination of 10%.

Measurement of Fat Mass Percentages by Bioimpedance Spectroscopy (BIS)

Mice were anesthetized with 1% isoflurane and lied flat dorsal side up with limbs perpendicular and tail straight. Four electrodes of ImpediVET were fastened with 28G needles and placed on four points on the midline of the body. Fat-free mass (FFM) and fat mass (FM) were calculated from total body water (TBW) determined by input body weight and complex impedance plotting of 256 frequencies between 4 kHz and 1000 kHz.

### Results

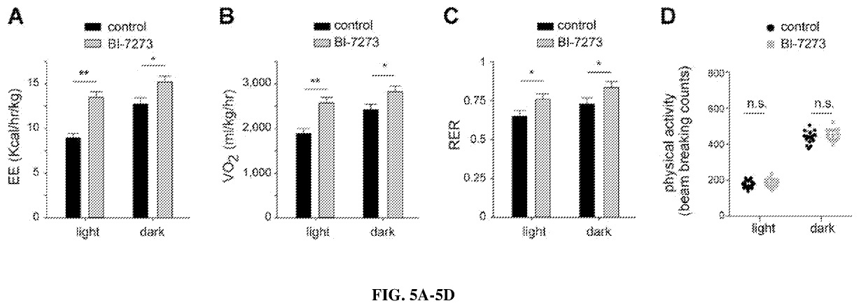

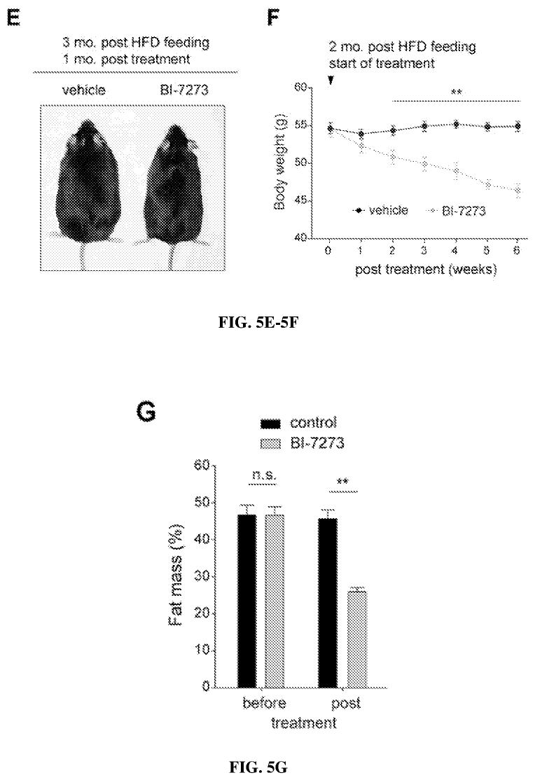

Next, whether oral administration of BI-7273 is sufficient to increase the whole-body energy expenditure was investigated. DIO models were gavaged with BI-7273 or control 0.5% Natrosol for 14 days as described above and later housed individually in metabolic cages for 10 days. Indirect calorimetry revealed that BI-7273 treatment drastically increased the total energy expenditure (E.E.) and oxygen consumption (VO₂) of DIO mice in both light and dark cycles (FIG. 5A, 5B), which is in concert with the induction of beige adipocytes in iWAT/eWAT in these mice. HFD-fed control DIO models had very low respiratory exchange ratios (RER; ˜0.6 in light cycles and 0.72 in dark cycles), which is consistent with their high lipid utilization and insulin resistance (IR) states. Intriguingly, BI-7273 treated DIO models had markedly increased RER in both light and dark cycles (FIG. 5C), indicating BI-7273 treatment increased carbohydrate utilization and reversed IR. The physical activity of control and BI-7273 treated DIO models were comparable (in either light or dark cycles) during the indirect calorimetry measurement (FIG. 5D), which rules out the possibility that the augmented energy expenditure in BI-7273 treated mice was due to increased physical activity.

Given the above augmentation of whole-body energy expenditure in BI-7273 treated DIO models, whether BI-7273 is sufficient to induce body weight loss and reduce adiposity (anti-obesity effects) was also investigated. DIO models were gavaged with BI-7273 or control 0.5% Natrosol for 14 days as described above and later fed with HFD for one additional month. One month after gavage, BI-7273 treated mice had an apparent leaner shape than control mice (FIG. 5E). Continuous measurement of body weight in both groups indicated that BI-7273 oral administration induced significant weight loss starting at the end of the 2-week gavaging and last at least for four additional weeks (mice were euthanized one month after gavaging; FIG. 5F). On average, BI-7273 treated DIO mice lost ˜15.5% of body weight at the end of the study (1.5 months after the start of gavage; FIG. 5F). This weight loss rate is higher than the criterium established by the Endocrine Society for effective treatment of obesity (>5% weight loss over three months) (Apovian, C. M., et al., J Clin Endocrinol Metab, 100, 342-362 (2015)).

Using bioimpedance spectroscopy (BIS), the adiposity (fat mass percent) of mice before and after gavage was measured. Before gavage, DIO models in both groups had comparable fat mass percentages (˜47%); BI-7273 gavage markedly reduced the adiposity to ˜26% at the end of the study (1.5 months after the start of gavage), whereas 0.5% Natrosol did not alter the adiposity (FIG. 5G). On average, the reduced fat mass (55 grams×21%=11.6 grams) in BI-7273 treated mice was more than the body weight loss at the end of the study (˜9 grams), which indicates a concomitant increase of lean mass in BI-7273 treated DIO mice. Before and after gavage, DIO models in both groups had comparable food intake (FIG. 5H), indicating the weight loss after BI-7273 gavage was not due to reduced energy intake. Interestingly, BI-7273 treatment also improved treadmill performance (longer time until exhaustion; FIG. 5I), which is in concert with the reduced fat mass and increased lean mass in BI-7273 treated mice.

Overall, the above data indicate that oral administration of BI-7273 elicited anti-obesity effects in DIO mouse models by augmenting energy expenditure.

### Example 6: Oral Administration of BI-7273 Improves Insulin Sensitivity and Protected from Diabetic Hyperglycemia

### Materials and Methods

Measurement of HbA1c by DCA Vantage Analyzer

Blood samples were taken from the tail vein and measured on HbA1c cartridges (5035C) loaded in a DCA Vantage Analyzer (Siemens).

Intraperitoneal Glucose Tolerance Test (IPGTT)

IPGTT was performed after 16 hrs fasting. Glucose (2 g kg⁻¹ body weight) was injected intraperitoneally. Blood samples were taken from the tail vein. Glucose levels were measured at 0, 15, 30, 60, and 120 min after glucose injection using a glucometer (OneTouch, Ultra 2).

Intraperitoneal Insulin Tolerance Test (IPITT)

IPITT was performed after 6 hrs fasting. Insulin (0.75 U kg⁻¹ body weight, Roche Risch-Rotkreuz, Switzerland) was injected intraperitoneally. Blood samples were taken from the tail vein. Glucose levels were measured at 0, 15, 30, 60, and 120 min after insulin injection using a glucometer (OneTouch, Ultra 2).

### Results

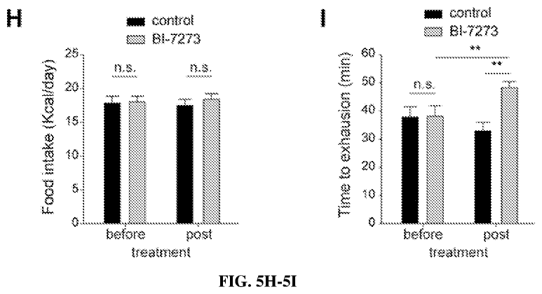

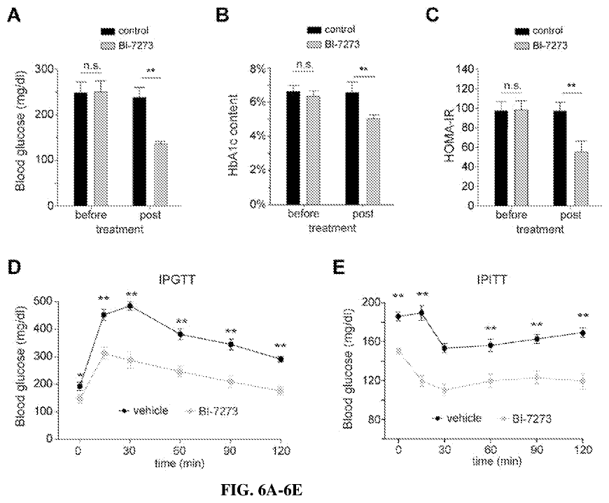

Next, whether BI-7273 oral administration is sufficient to protect DIO models from type 2 diabetes was investigated. Before gavage (3 months after HFD feeding), DIO models developed diabetes as evidenced by 1) averaged random non-fasting blood glucose level at ˜250 mg/dL; and 2) averaged glycated hemoglobin (HbA1c) level at 6.5% (FIG. 6A, B). BI-7273 oral administration (100 mg/kg/day, for 14 days) significantly lowered the random non-fasting blood glucose level (to 130 mg/dL) and the HbA1c level (˜5%; FIG. 6A, 6B). Upon HFD feeding, C57BL/6 mice are predisposed to type 2 diabetes, which is characterized by insulin resistance (averaged HOMA-IR index=95; FIG. 6C)(32). BI-7273 oral administration lowered HOMA-IR index (to ˜55%; FIG. 6C), indicating much improved β-cell function and insulin resistance. Besides, IPGTT and IPITT tests confirmed the drastically improved glucose tolerance and insulin sensitivity in BI-7273-treated type 2 diabetic DIO mice (FIG. 6D, 6E). Therefore, the above data indicate that oral administration of BI-7273 elicited anti-diabetes effects in DIO mouse models.

The experiments described herein reveal previously unappreciated roles of Brd9, Ankib1, Cacng1 and Gtl3 in repressing beige adipocyte lineage determination. BRD9 represses PGC1β expression and thus prevents mitochondrial biogenesis during beige adipogenic differentiation. Ablation or inhibition of BRD9 in white adipose progenitor cells increases beige adipogenic differentiation. Oral administration of a BRD9 inhibitor, BI-7273, induces extensive WAT beiging in diet-induced obese and diabetic mice. As a result, the mice have increased energy expenditure as well as improved glucose tolerance and insulin sensitivity.

Recent studies started to indicate that WAT beiging is under the influence of multiple factors (Harms, et al., Nat Med, 19, 1252-1263 (2013)). However, an unbiased screening for beige adipocyte inducers/repressors has not been reported by far. In this study, the genome-scale CRISPR Knock-Out (GeCKO) v2 pooled libraries were used to screen lineage repressors that prevent the beige adipogenic differentiation of white adipose progenitor cells. Given the recent advance in computational design/screening of small molecule inhibitors, the identification of such beige lineage repressors has profound implications in developing anti-obesity/anti-diabetes therapeutics. The GeCKO v2 pooled libraries contain ˜130,000 sgRNAs, which theoretically may target virtually all annotated protein-coding genes as well as many non-coding genes (Sanjana, N. E., et al., Nat Methods 11, 783-784 (2014)). During the screening, sgRNA-expressing lentiviruses had equal probabilities to infect cultured white adipose progenitor cells; meanwhile, at the population level, each gene target was targeted by multiple sgRNAs. Therefore, this screening approach holds promise to identify the most effective gene targets and sgRNAs in inducing beige adipocyte differentiation. Here, GFP fluorescence was used as a reporter to select a small number of beige adipocytes from the vast majority of white adipocytes in the mixed cell cultures. The GFP expression was induced by Cre-mediated recombination and the expression of Cre recombinase was in turn driven by a well-defined Ucp1 promoter (Nedergaard, J., et al., Biochim Biophys Acta, 1740, 293-304 (2005)). Notably, the Ucp1 promoter activity is responsive to early events during beige vs. white adipocyte lineage switching (e.g., the expression of lineage determinants Prdm16, PGC1α/β activates this promoter). On the other hand, the expression of GFP reporter, once induced by Cre-mediated recombination, was constitutively driven by a CAG promoter and no longer affected by the differentiation or thermogenic states of the cell. Thus, the screening design utilized herein is likely more sensitive to identify beige adipocyte lineage determinants than designs that are reliant on other functional modalities in beige adipocytes. Experiments validated the efficacy of this screening strategy in identifying UCP1^{+pos} thermogenic cells in mixed cell culture.

After the primary screening and the subsequent stringent bioinformatic selection, secondary screening was carried out for 14 sgRNAs targeting 10 candidate genes. The secondary screening confirmed 4 beige adipocyte lineage repressors (Brd9, Ankib1, Cacng1, Gtl3) out of the 10 candidate genes. This high success rate strongly supports the discriminatory power and efficiency of this screening-based approach. None of the four confirmed beige repressors has been reported to regulate beige adipocyte lineage determination. Brd9 (bromodomain containing 9) encodes a component of SWI/SNF/BAF chromatin remodeling complexes, which play important roles in regulating nucleosome positioning and transcription (Kadoch, C., et al., Nat Genet, 45, 592-601 (2013)). Ankib1 (ankyrin repeat and IBR domain containing 1) encodes a newly identified putative E3 ubiquitin ligase (Miller, S. L., et al., J Biol Chem, 279, 33528-33537 (2004)). Cacng1 (calcium voltage-gated channel auxiliary subunit gamma 1) encodes the gamma subunit of the skeletal muscle and neuronal dihydropyridine-sensitive calcium channel (Powers, P. A., et al., J Biol Chem, 268, 9275-9279 (1993)). Gtl3 (gene trap locus 3, a.k.a Cfap20 or Bug22) encodes protein related to tubulin post-translational modifications and cilium shape (Laligne, C., et al., Eukaryot Cell, 9, 645-655(2010); and Mendes Maia, T., et al., Biol Open, 3, 138-151 (2014)).

Studies show that transcriptional and epigenetic control is at the center of the beige adipocyte lineage determination (Inagaki, T., et al., Nat Rev Mol Cell Biol, 17, 480-495 (2016)). The transcriptional activation of Ucp1 is an important step in the development of thermogenic brown/beige adipocytes. In brown adipocytes, PGC1α, Prdm16, IRF4, thyroid hormone receptor and MED1 form a transcriptional activation complex, which is recruited to the regulatory enhancer regions of the Ucp1 gene under cold exposure (Chen, W., et al., Mol Cell, 35, 755-768 (2009); and Kong, X., et al., Cell, 158, 69-83 (2014)). Although PGC1a plays an important role in the transcriptional activation of Ucp1 during cold-induced thermogenesis, PGC1α is dispensable for the lineage determination of BAT (Uldry, M., et al., Cell Metab, 3, 333-341 (2006)). It was believed that with the loss of PGC1α, PGC1β can support the mitochondrial biogenesis during BAT lineage determination (Uldry, M., et al., Cell Metab, 3, 333-341 (2006)). Intriguingly, PGC1β, but not PGC1α, is important for rosiglitazone-induced mitochondrial biogenesis during WAT beiging indicating an obligatory role of PGC1β in beige adipocyte lineage determination (Pardo, R., et al., PLoS One, 6, e26989 (2011)). Consistent with this notion, the experiments disclosed herein indicate that BRD9 directly associates with the proximal promoter of Pgc1β gene, and sgRNA-mediated Brd9 genetic ablation increases the Pgc1β mRNA levels in multiple tissues (adipose tissues, skeletal muscle, heart, and liver). These data indicate that BRD9 represses the beige adipocyte lineage determination by repressing PGC1β expression.

Studies have started to reveal profound impacts of histone modifications and dynamic changes of the enhancer-promoter proximity in transcriptional regulation (Marsman, J., et al., Biochim Biophys Acta, 1819, 1217-1227 (2012); and Villarroya, F., et al., Biochimie, 134, 86-92 (2017)). For instance, the activation of Ucp1 transcription requires the dynamic control of the enhancer-promoter proximity of Ucp1 gene, which is mediated via the direct interaction of Prdm16 and the mediator protein MED1 (Harms, M. J., et al., Genes Dev, 29, 298-307 (2015); and Iida, S., et al., Genes Dev 29, 308-321 (2015)). Notably, histone demethylase JMJD1A is recruited to the PPAR response elements of the Ucp1 promoter via interaction with SWI/SNF/BAF chromatin remodeling complexes (Abe, Y., et al., Nat Commun, 6, 7052 (2015)). JMJD1A demethylates the H3K9me2 marks on the Ucp1 promoter and elicits the chromatin looping, which brings PPARγ/Prdm16/PGC1a transcriptional activators on Ucp1 distal enhancers to the Ucp1 proximal promoter (Abe, Y., et al., Nat Commun, 6, 7052 (2015); Karamanlidis, G., et al., J Biol Chem, 282, 24660-24669 (2007); and Kajimura, S., et al., Nature, 460, 1154-1158 (2009)). BRD9 is a component of BAF complex—one of the two principal SWI/SNF chromatin remodeling complexes in mammalian cells (Wang, X., et al., Clin Cancer Res, 20, 21-27 (2014)). Thus, Brd9-containing BAF complex may recruit histone deacetylase (HDACs) or methyltransferases (HMTs) to the Pgc1β promoter and establish a local repressive chromatin structure that prevents the above-mentioned chromatin looping and Ucp1 transcriptional activation. SWI/SNF chromatin remodeling complexes, although ubiquitously expressed, play important roles in lineage specification (Hu, G., et al., Genome Res, 21, 1650-1658 (2011)). Lineage-specific transcriptional regulators MyoD and Olig2 are capable of recruiting SWI/SNF complexes to the promoters of muscle and oligodendrocyte lineage-specific genes, respectively (de la Sema, I. L., et al., Nat Genet, 27, 187-190 (2001); and Yu, Y., et al., Cell, 152, 248-261 (2013)), and thus PPARγ may recruit BRD9-containing BAF complex specifically to the promoter of PGC1β gene leading to the repression of PGC1,8 expression. Besides beige adipocyte lineage determination, PGC1β has also been shown to induce mitochondrial biogenesis in skeletal muscle and heart; conversely, a repressed PGC1β expression is a fundamental cause of pressure overload-induced heart hypertrophy and heart failure (Riehle, C., et al., Trends Cardiovasc Med, 22, 98-105 (2012); Moslehi, J., et al., Circ Res, 110, 1226-1237 (2012); and Jung, S., et al., Integr Med Res, 3, 155-160 (2014)). Thus, understanding the repressive mechanism of BRD9 on PGC1β expression also has clinical implications in aging, muscle atrophy, and heart failure.

In sum, these studies identified four repressors of beige adipocyte lineage determination identified by an unbias genome-wide CRISPRi screening. Further characterization of BRD9 revealed its important role in repressing PGC1β in multiple tissues. This study also demonstrated the therapeutic potential of a BRD9-selective inhibitor, BI-7273, in treating obesity and insulin resistance in mouse models.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of inducing or increasing white adipose tissue beiging in a subject in need thereof comprising administering to the subject an effective amount of an inhibitor of a Brd9, Ankib1, Cacng1, and/or Gt13 (Cfap20) gene or a gene product thereof to increase differentiation of white adipose progenitor cells into beige or brown adipose cells.

2. The method of claim 1, wherein the subject has or is at risk of developing a metabolic disorder, obesity, reduced endurance or physical activity, muscle loss, or cardiovascular disease.

3. The method of claim 2, wherein the metabolic disorder is selected from the group consisting of insulin resistance, Type 1 or 2 diabetes mellitus, insulin insensitivity, impaired fasting glycaemia, impaired glucose tolerance (IGT), dysglycemia, and metabolic syndrome.

4. The method of claim 1 wherein the inhibitor is administered in an effective amount to increase expression of one or more beige lineage markers.

5. The method of claim 4, wherein the beige lineage markers are selected from Pgc1α, Pgc1β, Ucp1, Cedia, Dio2, Elov13, Cox8b, and combinations thereof.

6. The method of claim 1, wherein the inhibitor is administered in an effective amount to increase one or more markers of mitochondrial biogenesis.

7. The method of claim 6, wherein the markers of mitochondrial biogenesis are selected from Pgc1β, Ndufb2, Sdha, Uqcrc2, Cox8b, Atp5a1, copies of mitochondrial genomic DNA, and combinations thereof.

8. The method of claim 1, wherein the inhibitor induces or increases weight loss, prevents weight gain, reduces fat mass, increases lean mass, increases energy expenditure, increases time to exhaustion, increases oxygen consumption, improves β-cell function, improves insulin resistance, improves glucose tolerance, improves insulin sensitivity, or a combination thereof in the subject.

9. The method of claim 1, wherein the inhibitor is a small molecule inhibitor.

10. The method of claim 9, wherein the inhibitor is an inhibitor of the Brd9 gene or a gene product thereof.

11. The method of claim 10, wherein the inhibitor is selected from the group consisting of LP99, I-BRD9, BI-7273, BI-9564, GNE-375, and combinations thereof.

12. The method of claim 11, wherein the inhibitor is BI-7273 alone or in combination with I-BRD9.

13. The method of claim 11, wherein the inhibitor is BI-9564 alone or in combination with I-BRD9.

14. The method of claim 12, wherein the subject has or is at risk of developing a metabolic disorder, obesity, reduced endurance or physical activity, muscle loss, or cardiovascular disease.

15. The method of claim 1, wherein the inhibitor is antisense molecules, siRNA, miRNA, aptamers, ribozymes, triplex forming molecules, RNAi, external guide sequences, or a gene editing composition that targets the Brd9, Ankib1, Cacng1, and/or Gt13 (Cfap20) gene or gene product thereof.

16. The method of claim 15, wherein the inhibitor is a gene editing composition that induces a single or double strand break at the Brd9, Ankib1, Cacng1, and/or Gt13 (Cfap20) genetic locus in the subject and reduces expression thereof.

17.-18. (canceled)

19. The method of claim 1, wherein the inhibitor reduces expression or activity of a Brd9 gene and/or a gene product thereof.

20. The method of claim 1, wherein the subject is a human subject.

21. A pharmaceutical composition comprising an effective amount of an inhibitor of a Brd9, Ankib1, Cacng1, and/or Gt13 (Cfap20) gene or a gene product thereof to induce or increase weight loss, prevent weight gain, reduce fat mass, increases lean mass, increase energy expenditure, increase time to exhaustion, increase oxygen consumption, improve β-cell function, improve insulin resistance, improve glucose tolerance, improve insulin sensitivity, or a combination thereof in a subject.

22.-34. (canceled)

35. A method of treating a condition, disorder, or disease comprising administering a subject in need thereof an effective amount of an inhibitor of a Brd9, Ankib1, Cacng1, and/or Gt13 (Cfap20) gene or a gene product thereof to treat one or more symptoms of the condition, disorder, or disease,
wherein the condition, disorder, or disease is a metabolic disorder, obesity, reduced endurance or physical activity, muscle loss, or cardiovascular disease.


SEQUENCE LISTING (56 sequences)

SEQ ID NO: 1 - DNA, 82 bp, Artificial Sequence
  synthetic polynucleotide
aatgatacggcgaccaccgagatctacactctttccctacacgacgctcttccgatcttcttgtggaaaggacgaaacaccg

SEQ ID NO: 2 - DNA, 91 bp, Artificial Sequence
  synthetic polynucleotide
caagcagaagacggcatacgagatacattggcgtgactggagttcagacgtgtgctcttccgatcttctactattctttcccctgcactgt

SEQ ID NO: 3 - DNA, 21 bp, Artificial Sequence
  synthetic polynucleotide
acttcgtgcaagaaatgctga

SEQ ID NO: 4 - DNA, 20 bp, Artificial Sequence
  synthetic polynucleotide
cgccatgtctctagtgatcc

SEQ ID NO: 5 - DNA, 18 bp, Artificial Sequence
  synthetic polynucleotide
gcatggtgccttcgctga

SEQ ID NO: 6 - DNA, 20 bp, Artificial Sequence
  synthetic polynucleotide
aacttgtgcaggttggatgg

SEQ ID NO: 7 - DNA, 20 bp, Artificial Sequence
  synthetic polynucleotide
ctgggcgtggaattcgatga

SEQ ID NO: 8 - DNA, 20 bp, Artificial Sequence
  synthetic polynucleotide
ccaggatgacaccaaaaccc

SEQ ID NO: 9 - DNA, 21 bp, Artificial Sequence
  synthetic polynucleotide
tcctgtaaaagcccggagtat

SEQ ID NO: 10 - DNA, 20 bp, Artificial Sequence
  synthetic polynucleotide
atacatccagctcgcccttt

SEQ ID NO: 11 - DNA, 20 bp, Artificial Sequence
  synthetic polynucleotide
gaaccatgaagccaacgact

SEQ ID NO: 12 - DNA, 20 bp, Artificial Sequence
  synthetic polynucleotide
ggtcaagatcaaggtcccca

SEQ ID NO: 13 - DNA, 20 bp, Artificial Sequence
  synthetic polynucleotide
atactggcagatgacgtccc

SEQ ID NO: 14 - DNA, 18 bp, Artificial Sequence
  synthetic polynucleotide
cagtgtggtgcacgtctc

SEQ ID NO: 15 - DNA, 21 bp, Artificial Sequence
  synthetic polynucleotide
tccgcgttctcatgtaggtct

SEQ ID NO: 16 - DNA, 20 bp, Artificial Sequence
  synthetic polynucleotide
cgattttggcatgactcgga

SEQ ID NO: 17 - DNA, 20 bp, Artificial Sequence
  synthetic polynucleotide
gcattacaacatgggtggga

SEQ ID NO: 18 - DNA, 19 bp, Artificial Sequence
  synthetic polynucleotide
gaccccatcttgctttgct

SEQ ID NO: 19 - DNA, 21 bp, Artificial Sequence
  synthetic polynucleotide
agacagactgggaaaacatcg

SEQ ID NO: 20 - DNA, 20 bp, Artificial Sequence
  synthetic polynucleotide
gccgactaaatcaagcaaca

SEQ ID NO: 21 - DNA, 20 bp, Artificial Sequence
  synthetic polynucleotide
gaagcgattctagggagcag

SEQ ID NO: 22 - DNA, 23 bp, Artificial Sequence
  synthetic polynucleotide
tctggattgttcttcactcttgg

SEQ ID NO: 23 - DNA, 20 bp, Artificial Sequence
  synthetic polynucleotide
ggtcgatgtctgctttcctc

SEQ ID NO: 24 - DNA, 22 bp, Artificial Sequence
  synthetic polynucleotide
tggcatctctgtgtcaaccatg

SEQ ID NO: 25 - DNA, 20 bp, Artificial Sequence
  synthetic polynucleotide
ttctccaggctctcctttcc

SEQ ID NO: 26 - DNA, 20 bp, Artificial Sequence
  synthetic polynucleotide
acacattccaccaccagctt

SEQ ID NO: 27 - DNA, 20 bp, Artificial Sequence
  synthetic polynucleotide
cagcacattttgggaaggca

SEQ ID NO: 28 - DNA, 19 bp, Artificial Sequence
  synthetic polynucleotide
gctctggtaggggcagtga

SEQ ID NO: 29 - DNA, 20 bp, Artificial Sequence
  synthetic polynucleotide
acttactacccggtgtccat

SEQ ID NO: 30 - DNA, 20 bp, Artificial Sequence
  synthetic polynucleotide
gcgaagttcacagtggttcc

SEQ ID NO: 31 - DNA, 20 bp, Artificial Sequence
  synthetic polynucleotide
tcatagctgtcgtacctggg

SEQ ID NO: 32 - DNA, 20 bp, Artificial Sequence
  synthetic polynucleotide
cgagtcgcagaaaagaagcc

SEQ ID NO: 33 - DNA, 21 bp, Artificial Sequence
  synthetic polynucleotide
tgaaccaaagttgaccaccag

SEQ ID NO: 34 - DNA, 21 bp, Artificial Sequence
  synthetic polynucleotide
ggacctgatgcaaccctatga

SEQ ID NO: 35 - DNA, 21 bp, Artificial Sequence
  synthetic polynucleotide
atgtctcaccttgctcctact

SEQ ID NO: 36 - DNA, 20 bp, Artificial Sequence
  synthetic polynucleotide
ttatctccaggcctgcaaga

SEQ ID NO: 37 - DNA, 23 bp, Artificial Sequence
  synthetic polynucleotide
ttggtaaactcaagatcctgagg

SEQ ID NO: 38 - DNA, 20 bp, Artificial Sequence
  synthetic polynucleotide
ggagtagggagccaagtact

SEQ ID NO: 39 - DNA, 20 bp, Artificial Sequence
  synthetic polynucleotide
caatgggcataaagctatgg

SEQ ID NO: 40 - DNA, 21 bp, Artificial Sequence
  synthetic polynucleotide
ggagcagcgattctgagtaga

SEQ ID NO: 41 - DNA, 20 bp, Artificial Sequence
  synthetic polynucleotide
ctattaaaaagtaggctggg

SEQ ID NO: 42 - DNA, 21 bp, Artificial Sequence
  synthetic polynucleotide
gctggcttcagtcacagtgtg

SEQ ID NO: 43 - DNA, 18 bp, Artificial Sequence
  synthetic polynucleotide
ccctagactcttgggttc

SEQ ID NO: 44 - DNA, 19 bp, Artificial Sequence
  synthetic polynucleotide
ggcaccctgaagccatgag

SEQ ID NO: 45 - DNA, 20 bp, Artificial Sequence
  synthetic polynucleotide
agcgagcaagcgagcgagga

SEQ ID NO: 46 - DNA, 18 bp, Artificial Sequence
  synthetic polynucleotide
gagacctggtccctgcgc

SEQ ID NO: 47 - DNA, 17 bp, Artificial Sequence
  synthetic polynucleotide
gctccggcagccaggtg

SEQ ID NO: 48 - DNA, 21 bp, Artificial Sequence
  synthetic polynucleotide
gcctcagtttccccagctgtg

SEQ ID NO: 49 - DNA, 20 bp, Artificial Sequence
  synthetic polynucleotide
cccgtcactcaaccatttca

SEQ ID NO: 50 - DNA, 20 bp, Artificial Sequence
  synthetic polynucleotide
cttatcaatgggggctctgg

SEQ ID NO: 51 - DNA, 20 bp, Artificial Sequence
  synthetic polynucleotide
agatatggctggctttgtgc

SEQ ID NO: 52 - DNA, 19 bp, Artificial Sequence
  synthetic polynucleotide
gaactcgctcaggttctgc

SEQ ID NO: 53 - DNA, 25 bp, Artificial Sequence
  synthetic polynucleotide
caccgagataccgtgtactacaagt

SEQ ID NO: 54 - DNA, 25 bp, Artificial Sequence
  synthetic polynucleotide
caccgagggagcactgtgacacgga

SEQ ID NO: 55 - DNA, 85 bp, Artificial Sequence
  synthetic polynucleotide
aatgatacggcgaccaccgagatctacactctttccctacacgacgctcttccgatctttttcttgtggaaaggacgaaacaccg

SEQ ID NO: 56 - DNA, 88 bp, Artificial Sequence
  synthetic polynucleotide
aatgatacggcgaccaccgagatctacactctttccctacacgacgctcttccgatctaaaaaatcttgtggaaaggacgaaacaccg